(19) United States
(12) Patent Application Publication
(10) Pub. No.: US 2008/0243213 A1
(43) Pub. Date: Oct. 2, 2008

(54) CURATIVE TREATMENT SYSTEM, CURATIVE TREATMENT DEVICE, AND TREATMENT METHOD FOR LIVING TISSUE USING ENERGY

(76) Inventors: Tomoyuki TAKASHINO, Hino-shi (JP); Toru NAGASE, Tachikawa-shi (JP); Koji IIDA, Sagamihara-shi (JP); Mai WAKAMATSU, Hino-shi (JP)

Correspondence Address: OSTROLENK FABER GERB & SOFFEN, 1180 AVENUE OF THE AMERICAS, NEW YORK, NY, 100368403, US

(21) Appl. No.: 11/695,140

(22) Filed: Apr. 2, 2007

(51) Int. Cl. A61N 1/02

(52) U.S. Cl. 607/115

(57) ABSTRACT

A treatment system that applies energy to a living tissue includes first and second holding members, an operating section, an energy source, and a plurality of energy applying portions that apply energy supplied from the energy source. Each of the first and second holding members has a holding surface to hold the living tissue. The operating section operates a relative movement of at least one of the first and second holding members with respect to the other. The energy source supplies energy to at least one of the first and second holding members. The plurality of energy applying portions are provided on the applying surface of at least one of the first and second holding members, and uniforms density of energy applied to a living tissue held by the first and second holding members.

### BACKGROUND OF THE INVENTION

1. Field of the Invention

The present invention relates to a treatment system, a treatment device, and a treatment method for a living tissue using energy that enable energy to function with respect to a living tissue in a state where the living tissue is held.

2. Description of the Related Art

US Patent Application Publication No. 2005/0113828 A1 discloses electro-surgical instruments including a pair of juxtaposed jaw members each having an electroconductive surface. An over-shoe having a plurality of apertures is arranged in the pair of jaw members of the electro-surgical instruments. The over-shoe has, e.g., insulating properties. Therefore, energy for a treatment is supplied to a living tissue from the jaw members through the apertures of the over-shoe. Further, the apertures of the over-shoe are arranged in two rows along a longitudinal direction of the over-shoe.

### BRIEF SUMMARY OF THE INVENTION

According to a first aspect of the present invention, there is provided a treatment system that applies energy to a living tissue, the system includes:

first and second holding members each having a holding surface to hold the living tissue;

an operating section that operates a relative movement of at least one of the first and second holding members with respect to the other;

an energy source that supplies energy to at least one of the first and second holding members; and

a plurality of energy applying portions that apply energy supplied from the energy source, the plurality of energy applying portions being provided on the holding surface of at least one of the first and second holding members and uniformly controlling density of energy applied to the living tissue held by the first and second holding members.

According to a second aspect of the present invention, there is provided a treatment device that allows energy to function with respect to a living tissue, the device includes:

a holding section that holds the living tissue, the holding section including:

first and second holding members that are relatively movable with respect to each other; and

a plurality of energy applying portions that are provided on at least one of the first and second holding members and connected with an energy source, the energy applying portions being provided on at least one of the first and second holding members and uniforming density of energy applied to the living tissue when applying the energy to the living tissue held by the first and second holding members.

According to a third aspect of the present invention, there is provided a treatment method for a living tissue using energy, the method includes:

holding the living tissue;

applying energy to the living tissue and denaturing the living tissue; and

uniforming energy density at a desired position where the held living tissues denatures by the energy applied to the living tissue.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic view showing a treatment system according to a first embodiment of the present invention;

FIG. 1B is a schematic view when the treatment system according to the first embodiment is used to perform a bipolar type treatment;

FIG. 2A is a schematic longitudinal sectional view showing a shaft and a state where a first holding member and a second holding member of a holding section in an electro-surgical device according to the first embodiment are closed;

FIG. 2B is a schematic longitudinal sectional view showing the shaft and a state where the second holding member of the holding section are opened with respect to the first holding member in the electro-surgical device according to the first embodiment;

FIG. 3A is a schematic plan view showing the first holding member on a side close to the second holding member in the holding section of the electro-surgical device according to the first embodiment;

FIG. 3B is a schematic longitudinal sectional view showing the first holding member taken along a line 3B-3B depicted in FIG. 3A in the holding section of the electro-surgical device according to the first embodiment;

FIG. 3C is a schematic cross sectional view cut along the 3C-3C line of FIG. 3A, showing the first holding member in the holding section of the electro-surgical device according to the first embodiment;

FIG. 4A is a schematic view showing a surface of a main body of the first holding member in the holding section of the electro-surgical device according to the first embodiment and temperature distribution of a living tissue when energy is applied to the living tissue from electrodes on the surface of the main body of the first holding member;

FIG. 4B is a schematic view showing a prior art for comparison with the schematic view showing the surface of the main body of the first holding member in the holding section of the electro-surgical device according to the first embodiment and the temperature distribution of the living tissue when energy is applied to the living tissue from the electrodes on the surface of the main body of the first holding member depicted in FIG. 4A;

FIG. 5A is a schematic view when a treatment system according to the first embodiment is used to perform a bipolar type treatment;

FIG. 5B is a schematic view when the treatment system according to the first embodiment is used to perform a monopolar type treatment;

FIG. 5C is a schematic view when the treatment system according to the first embodiment is used to perform a monopolar type treatment;

FIG. 6 is a schematic view showing a modification of the treatment system according to the first embodiment of the present invention;

FIG. 7A is a schematic view showing a surface of a main body of a first holding member in a holding section of an electro-surgical device according to a second embodiment of the present invention and temperature distribution of a living tissue when energy is applied to the living tissue from electrodes on the surface of the main body of the first holding member;

FIG. 7B is a schematic view showing a prior art for comparison with the schematic view showing the surface of the main body of the first holding member in the holding section of the electro-surgical device according to the second embodiment and the temperature distribution of the living tissue when energy is supplied to the living tissue from the electrodes on the surface of the main body of the first holding member depicted in FIG. 7A;

FIG. 8 is a schematic plan view showing a first holding member on a side close to a second holding member in a holding section of an electro-surgical device according to a third embodiment of the present invention;

FIG. 9 is a schematic plan view showing a first holding member on a side close to a second holding member in a holding section of an electro-surgical device according to a fourth embodiment of the present invention;

FIG. 10 is a schematic plan view showing a first holding member on a side close to a second holding member in a holding section of an electro-surgical device according to a fifth embodiment of the present invention;

FIG. 11 is a schematic plan view showing a first holding member on a side close to a second holding member in a holding section of an electro-surgical device according to a sixth embodiment of the present invention;

FIG. 12 is a schematic view showing a treatment system according to a seventh embodiment of the present invention;

FIG. 13A is a schematic longitudinal sectional view showing a shaft and a state where a first holding member and a second holding member of a holding section in an electro-surgical device according to the seventh embodiment are closed;

FIG. 13B is a schematic longitudinal sectional view showing the shaft and a state where the second holding member of the holding section are opened with respect to the first holding member in the electro-surgical device according to the seventh embodiment;

FIG. 14 is a schematic plan view showing the first holding member on a side close to the second holding member in the holding section of the electro-surgical device according to the seventh embodiment;

FIG. 15 is a schematic view showing a treatment system according to an eighth embodiment of the present invention;

FIG. 16A is a schematic longitudinal sectional view showing a state where a main body side holding section is engaged with a detachable side holding section so that the detachable side holding section is separated from the main body side holding section in an electro-surgical device according to the eighth embodiment;

FIG. 16B is a schematic longitudinal sectional view showing a state where the main body side holding section is engaged with the detachable side holding section so that the detachable side holding section is close to the main body side holding section in the electro-surgical device according to the eighth embodiment;

FIG. 16C is an enlarged schematic longitudinal sectional view showing a part of the main body side holding section denoted by reference character 16C in the electro-surgical device according to the eighth embodiment depicted in FIG. 16A;

FIG. 16D is an enlarged schematic longitudinal sectional view showing a part of the detachable side holding section denoted by reference character 16D in the electro-surgical device according to the eighth embodiment depicted in FIG. 16A;

FIG. 17 is a schematic view showing the main body side holding section in the electro-surgical device according to the eighth embodiment and temperature distribution of a living tissue when energy is supplied to the living tissue from electrodes on a surface of the main body side holding section;

FIG. 18A is a schematic view showing the surface of the main body side holding section in the holding section of the electro-surgical device according to the eighth embodiment and temperature distribution of a living tissue when energy is applied to the living tissue from electrodes on the surface of the main body side holding section;

FIG. 18B is a schematic view showing a prior art for comparison with the schematic view showing the surface of the main body side holding section in the holding section of the electro-surgical device according to the eighth embodiment and the temperature distribution of the living tissue when energy is applied to the living tissue from the electrodes on the surface of the main body side holding section depicted in FIG. 18A;

FIG. 19 is a schematic view showing a main body side holding section in an electro-surgical device according to a ninth embodiment and temperature distribution of a living tissue when energy is applied to the living tissue from electrodes on a surface of the main body side holding section;

FIG. 20 is a schematic view showing a main body side holding section in an electro-surgical device according to a tenth embodiment and temperature distribution of a living tissue when energy is applied to the living tissue from electrodes on a surface of the main body side holding section;

FIG. 21 is a schematic view showing a main body side holding section in an electro-surgical device according to an eleventh embodiment and temperature distribution of a living tissue when energy is applied to the living tissue from electrodes on a surface of the main body side holding section; and

FIG. 22 is a schematic view showing a main body side holding section in an electro-surgical device according to a twelfth embodiment and temperature distribution of a living tissue when energy is applied to the living tissue from electrodes on a surface of the main body side holding section.

### DETAILED DESCRIPTION OF THE INVENTION

The best mode for carrying out the present invention will now be explained hereinafter with reference to the accompanying drawings.

### First Embodiment

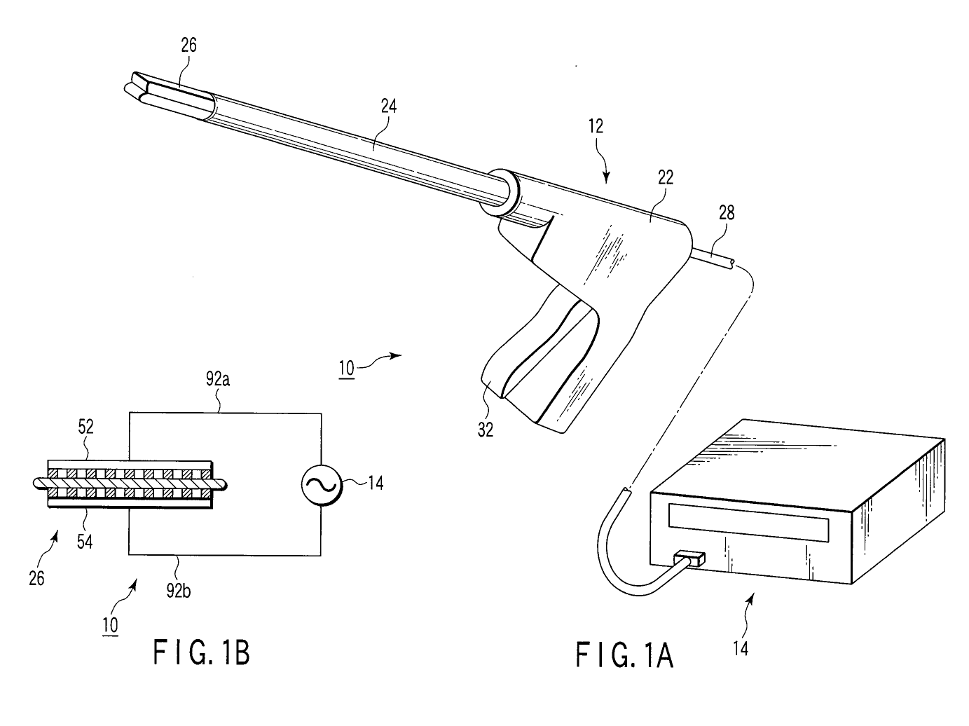

A first embodiment will be explained with reference to FIGS. 1 to 6.

Here, as an example of an energy treatment device, a linear type bipolar electro-surgical device 12 which performs a treatment through, for example, an abdominal wall will be described.

As shown in FIGS. 1A and 1B, a treatment system 10 includes the electro-surgical device (a treatment device for curing) 12 and an energy source 14.

The electro-surgical device 12 includes a handle 22, a shaft 24 and an openable/closeable holding section 26. The handle 22 is connected with the energy source 14 via a cable 28. The energy source 14 is connected to a foot switch and a handle switch (not shown). Therefore, these foot and hand switches are operated by an operator to switch ON/OFF of the supply of energy from the energy source 14 to the electro-surgical device 12.

The handle 22 is substantially formed into an L-shape. The shaft 24 is disposed on one end of the handle 22. The cable 28 is extended from a proximal side of the handle 22 disposed coaxially with the shaft 24.

On the other hand, the other end of the handle 22 is a grip held by the operator. The handle 22 includes a holding section opening/closing knob 32 arranged on the other end of the handle 22. The holding section opening/closing knob 32 is connected to a proximal end of a sheath 44 described later of the shaft 24 substantially at the center of the handle 22. When the holding section opening/closing knob 32 is allowed to come close to or come away from the other end of the handle 22, the sheath 44 moves along an axial direction of the shaft 24.

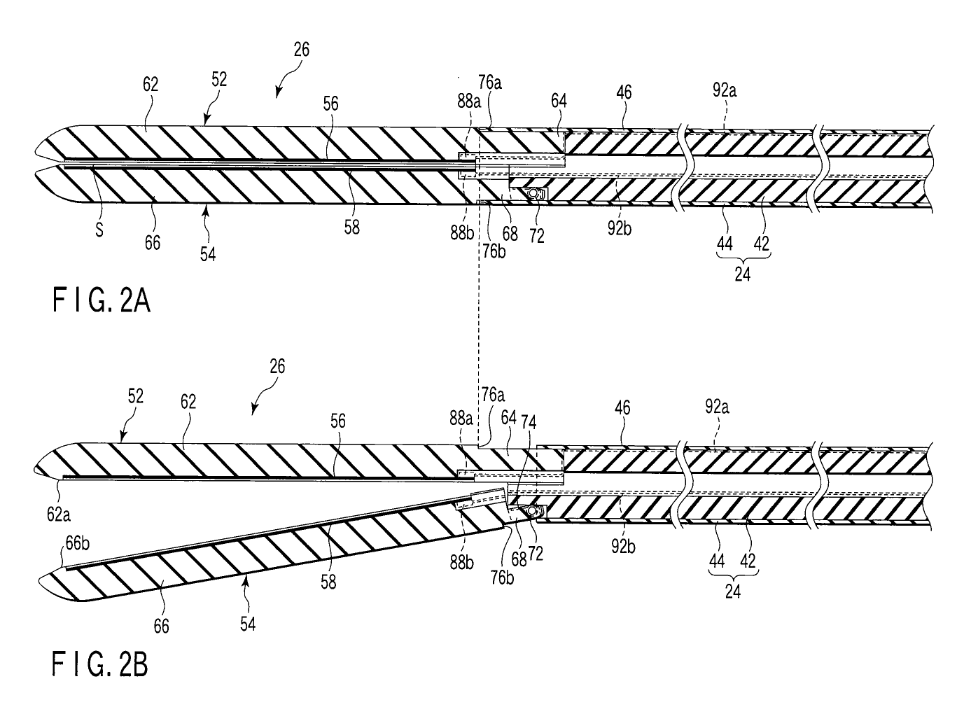

As shown in FIGS. 2A and 2B, the shaft 24 includes a cylindrical member 42 and the sheath 44 slidably disposed outside the cylindrical member 42. A proximal end of the cylindrical member 42 is fixed to the handle 22. The sheath 44 is slidable along an axial direction of the cylindrical member 42.

Outside the cylindrical member 42, concave portion 46 is formed along the axial direction of the cylindrical member 42. The concave portion 46 is provided with a first conducting line 92a connected to a first high-frequency electrode 56 described later. A second conducting line 92b connected to a second high-frequency electrode 58 described later is passed through the cylindrical member 42.

It is to be noted that the first high-frequency electrode plate 56 is electrically connected with a first electrode connector 88a. The first electrode connector 88a is connected with the cable 28 extended from the handle 22 via a first energization line 92a. The second high-frequency electrode plate 58 is electrically connected with a second electrode connector 88b. The second electrode connector 88a is connected with the cable 28 extended from the handle 22 via a second energization line 92b.

As shown in FIGS. 1A, 2A, and FIG. 2B, the holding section 26 is disposed at a distal end of the shaft 24. As shown in FIGS. 2A and 2B, the holding section 26 includes a first holding portion 52, a second holding portion 54, the first high-frequency electrode 56 as an output portion or an energy applying portion, and the second high-frequency electrode 58 as another output portion or another energy release portion.

It is preferable that the first holding portion 52 and the second holding portion 54 entirely have insulating properties, respectively. The first holding portion 52 integrally includes a first holding portion main body (hereinafter referred to mainly as the main body) 62 provided with the first high-frequency electrode 56 and a base portion 64 disposed at a proximal end of the main body 62. The second holding portion 54 integrally includes a second holding portion main body 66 provided with the second high-frequency electrode 58 and a base portion 68 disposed at a proximal end of the main body 66.

The base portion 64 of the first holding portion 52 is fixed to a distal end of the cylindrical member 42 of the shaft 24. On the other hand, the base portion 68 of the second holding portion 54 is rotatably supported at the distal end of the cylindrical member 42 of the shaft 24 by a support pin 72 disposed in a direction crossing the axial direction of the shaft 24 at right angles. The second holding portion 54 can rotate around an axis of the support pin 72 to open or close with respect to the first holding portion 52. Moreover, the second holding portion 54 is urged so as to open with respect to the first holding portion 52 by an elastic member 74 such as a leaf spring.

Outer surfaces of the main bodies 62 and 66 of the first holding portion 52 and the second holding portion 54 are formed into smooth curved surfaces. Similarly, outer surfaces of the base portions 64 and 68 of the first holding portion 52 and the second holding portion 54 are also formed into smooth curved surfaces. While the second holding portion 54 is closed with respect to the first holding portion 52, sections of the main bodies 62, 66 of the support members 52, 54 are formed into substantially circular or elliptic shapes. When the second holding portion 54 is closed with respect to the first holding portion 52, the base portions 64, 68 are formed into cylindrical shapes. In this state, a diameter of each of the proximal ends of the main bodies 62, 66 of the first holding portion 52 and the second holding portion 54 is formed to be larger than a diameter of each of the base portions 64, 68. Moreover, stepped portions 76a, 76b are formed between the main bodies 62, 66 and the base portions 64, 68, respectively.

Here, in the first holding portion 52 and the second holding portion 54, while the second holding portion 54 is closed with respect to the first holding portion 52, a substantially circular or elliptic outer peripheral surface formed by combining the base portions 64, 68 of the holding portions 52, 54 is substantially the same plane as that of an outer peripheral surface of the distal end of the cylindrical member 42, or a diameter of the outer peripheral surface is formed to be slightly larger than that of the outer peripheral surface of the distal end of the cylindrical member 42. Therefore, the sheath 44 can be slid with respect to the cylindrical member 42 to cover the base portions 64, 68 of the first holding portion 52 and the second holding portion 54 with a distal end of the sheath 44. In this state, as shown in FIG. 2A, the first holding portion 52 and the second holding portion 54 close against an urging force of the elastic member 74. On the other hand, the sheath 44 is slid toward the proximal end of the cylindrical member 42 from the state in which the base portions 64, 68 of the first holding portion 52 and the second holding portion 54 are covered with the distal end of the sheath 44. In this case, as shown in FIG. 2B, the second holding portion 54 is opened with respect to the first holding portion 52 by the urging force of the elastic member 74.

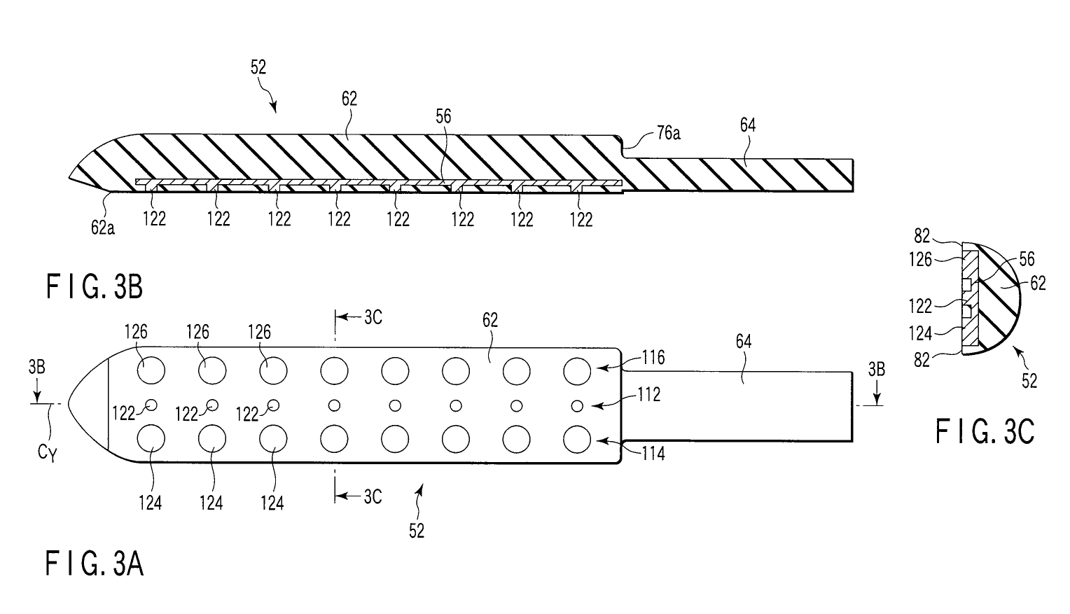

As shown in FIGS. 3B and 3C, the first high-frequency electrode plate 56 is arranged in the main body 62 of the first holding member 52. As shown in FIG. 3A, the first high-frequency electrode plate 56 includes a first high-frequency electrode group (which will be referred to as a first electrode group hereinafter) 112, a second high-frequency electrode group (which will be referred to as a second electrode group hereinafter) 114, and a third high-frequency electrode group (which will be referred to as a third electrode group hereinafter) 116 in each of columns. As shown in FIG. 3B, the first electrode group 112, the second electrode group 114, and the third electrode group 116 include a plurality of (eight in each group in this example) electrodes 122, 124, and 126 each having a convex cross section along a longitudinal direction of the main body 62 like spots.

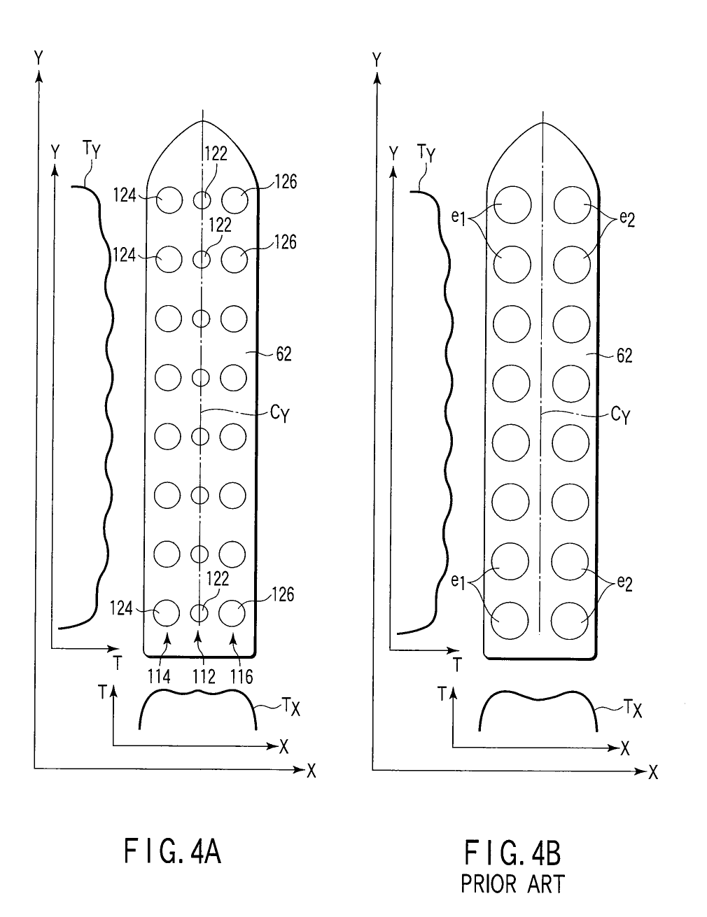

The first electrode group 112 is arranged in a region (a first region) along a central axis C_{Y} of the main body 62 in the longitudinal direction (a Y axis direction in FIG. 4A). The second electrode group 114 is arranged in a region (a second region) away from the central axis C_{Y} of the main body 62 by a predetermined distance. Likewise, the third electrode group 116 is arranged in a region (the second or third region) away from the central axis C_{Y} of the main body 62 by a predetermined distance. That is, the first electrode group 112, the second electrode group 114, and the third electrode group 116 are respectively arranged in the Y axis direction in FIG. 4A.

It is to be noted that the second electrode group 114 and the third electrode group 116 are arranged at substantially symmetrical positions with respect to the central axis C_{Y} of the main body 62. That is, the second electrode group 114 and the third electrode group 116 are arranged at substantially symmetrical positions with respect to the first electrode group 112. In other words, a distance between the first electrode group 112 and the second electrode group 114 is substantially equal to a distance between the first electrode group 112 and the third electrode group 116. Further, one electrode 122 of the first electrode group 112, one electrode 124 of the second electrode group 114, and one electrode 126 of the third electrode group 116 are arranged on the same axis in the X axis direction in FIG. 4A (see FIG. 3C).

Exposed areas of the respective electrodes 124 and 126 in the second electrode group 114 and the third electrode group 116 are substantially equal to each other. An exposed area of each electrode 122 in the first electrode group 112 is smaller than the exposed area of each of the electrodes 124 and 126 in the second electrode group 114 and the third electrode group 116. Further, a distance between the respective electrodes 122 in the first electrode group 112, a distance between the respective electrodes 124 in the second electrode group 114, and a distance between the respective electrodes 126 in the third electrode group 116 are substantially equal to each other.

Here, it is assumed that outputs from the respective electrodes 122, 124, and 126 in the first to the third electrode groups 112, 114, and 116 per unit area are in proportion to each other.

Furthermore, the second high-frequency electrode plate 58 is also arranged on the second holding member 54 to be symmetrical to the first holding member 52. A detailed explanation of this structure will be omitted.

A function of a treatment system 10 according to this embodiment will now be explained.

As shown in FIG. 2A, in a state where the second holding member 54 is closed with respect to the first holding member 52, the holding section 26 and the shaft 24 of the electro-surgical device 12 are inserted into, e.g., an abdominal cavity through an abdominal wall. The holding section 26 of the electro-surgical device 12 is opposed to a living tissue as a treatment target.

The holding section opening/closing knob 32 of the handle 22 is operated to hold the living tissue as a treatment target by using the first holding member 52 and the second holding member 54. At this time, the sheath 44 is moved to a proximal end side of the shaft 24 with respect to the cylindrical body 42. A space between the base portions 64 and 68 cannot be maintained in a cylindrical shape due to an urging force of the elastic member 74, and the second holding member 54 is then opened with respect to the first holding member 52.

Moreover, the living tissue as a treatment target is arranged between the first high-frequency electrode plate 56 of the first holding member 52 and the second high-frequency electrode plate 58 of the second holding member 54. In this state, the holding section opening/closing knob 32 of the handle 22 is operated. At this time, the sheath 44 is moved to a distal end side of the shaft 24 with respect to the cylindrical body 42. The base portions 64 and 68 are closed to form the cylindrical shape therebetween against the urging force of the elastic member 74 by using the sheath 44. Therefore, the first holding member main body 62 integrally formed on the base portion 64 and the second holding member main body 66 integrally formed on the base portion 68 are closed. That is, the second holding member 54 is closed with respect to the first holding member 52. Therefore, the living tissue as a treatment target is held between the first holding member 52 and the second holding member 54.

At this time, the living tissue as a treatment target is in contact with both the electrodes 122, 124, and 126 of the first high-frequency electrode plate 56 provided on the first holding member 52 and the electrodes 122, 124, and 126 of the second high-frequency electrode plate 58 provided on the second holding member 54. A surrounding tissue of the living tissue as a treatment target is appressed against both a contact surface of the edge portion 82 of the first holding member and a contact surface of the edge portion (not shown) of the second holding member 54.

In this state, the foot switch or the hand switch is operated. Energy is respectively supplied to the first high-frequency electrode plate 56 and the second high-frequency electrode plate 58 from the energy source 14 through the cable 28, the first and second energization lines 92a and 92b, and the first and second energization connectors 88a and 88b.

Since the treatment system 10 according to the embodiment is of a bipolar type as shown in FIGS. 1A and 1B, the electrodes 122, 124, and 126 of the first high-frequency electrode plate 56 apply a high-frequency current to a space between themselves and the electrodes 122, 124, and 126 of the second high-frequency electrode plate 58 via the living tissue as a treatment target. Therefore, the living tissue held between the main body 62 of the first holding member 52 and the main body 66 of the second holding member 54 is heated.

As this time, as shown in FIGS. 3A and 4A, each electrode 122 in the first electrode group 112 has a smaller contact area with respect to the living tissue than that of each electrode 124 or each electrode 126 in the second electrode group 114 or the third electrode group 116. Therefore, energy applied to the living tissue from each electrode 122 in the first electrode 112 is smaller than energy applied to the living tissue from each electrode 124 and each electrode 126 in the second electrode group 114 and the third electrode 116.

On the other hand, the living tissue that is in contact with the second electrode group 114 or the third electrode group 116 is away from the central axis C_{Y} and close to the outside of the holding section 26. Therefore, the living tissue is affected by the outside of the holding section 26 having temperature far lower than that of the living tissue present between the first holding member 52 and the second holding member 54. However, the living tissue near the central axis C_{Y} of the main body 62 of the first holding member 52 is maintained at high temperature due to functions of the second and third electrode groups 114 and 116. Therefore, even if a heating power near the central axis C_{Y} that is applied from the first electrode group 112 is small, it further approximates a flat shape.

Therefore, temperature distribution (energy distribution or energy density) T_{X} of the living tissue when energy is applied to the living tissue from the first high-frequency electrode plate 56 on a surface (a holding surface 62a) of the main body 62 of the first holding member 52 in the X axis direction further approximates a flat surface from a position near the central axis C_{Y} to a position corresponding to an edge portion of the main body 62 of the first holding member 52 away from the central axis C_{Y}. That is, a temperature gradient of the living tissue in the holding section 26 along the X axis direction is reduced as much as possible.

Therefore, the living tissue is uniformly treated in the X axis direction of the holding section 26. Thus, when, e.g., welding the living tissue, the living tissue is uniformly cauterized, thereby obtaining uniform conjugation strength.

Meanwhile, electrodes e₁ and e₂ are arranged in two columns at positions away from a central axis C_{Y} by an equal distance on a main body 62 of a first holding member 52 according to the prior art depicted in FIG. 4B. When giving a treatment to a living tissue held by such a first holding member 52, temperature distribution T_{X} shown in FIG. 4B is demonstrated, for example. The temperature distribution T_{X} has a depression at a central part (near the central axis C_{Y}), and temperature of the living tissue at a position corresponding to an edge portion of the main body 62 of the first holding member 52 is also reduced. Therefore, performing a uniform treatment with respect to the living tissue is difficult.

As explained above, according to the embodiment, the following effect can be obtained.

As shown in FIG. 4A, the first electrode group 112 is arranged on the central axis C_{Y} of the main body 62 of the first holding member 52. Further, a contact area of each electrode 122 in the first electrode group 112 with respect to the living tissue is set to be smaller than those of the respective electrodes 124 and 126 in the second electrode group 114 and the third electrode group 116. That is, an amount of energy supplied to the living tissue from each electrode 122 in the first electrode group 112 is smaller than an amount of energy supplied to the living tissue from each electrode 124 or 126 in the second or the third electrode group 114 or 116.

Then, the temperature distribution T_{X} in the X axis direction given to the living tissue from the main body 62 of the first holding member 52 depicted in FIG. 4A can be uniformed from the central part (near the central axis C_{Y}) of the main body 62 of the first holding member 52 to a position corresponding to the edge part of the same as compared with the temperature distribution T_{X} of the prior art shown in FIG. 4B. That is, temperature gradient of the temperature distribution T_{X} in the X axis direction given to the living tissue by the main body 62 of the first holding member 52 depicted in FIG. 4A can be more flattened as compared with temperature gradient of the temperature distribution T_{X} of the prior art shown in FIG. 4B. Then, an arrangement of the electrodes 122, 124, and 126 in the X axis direction of the main body 62 of the first holding member 52 enables performing a uniform treatment, e.g., weld or cautery with respect to the living tissue.

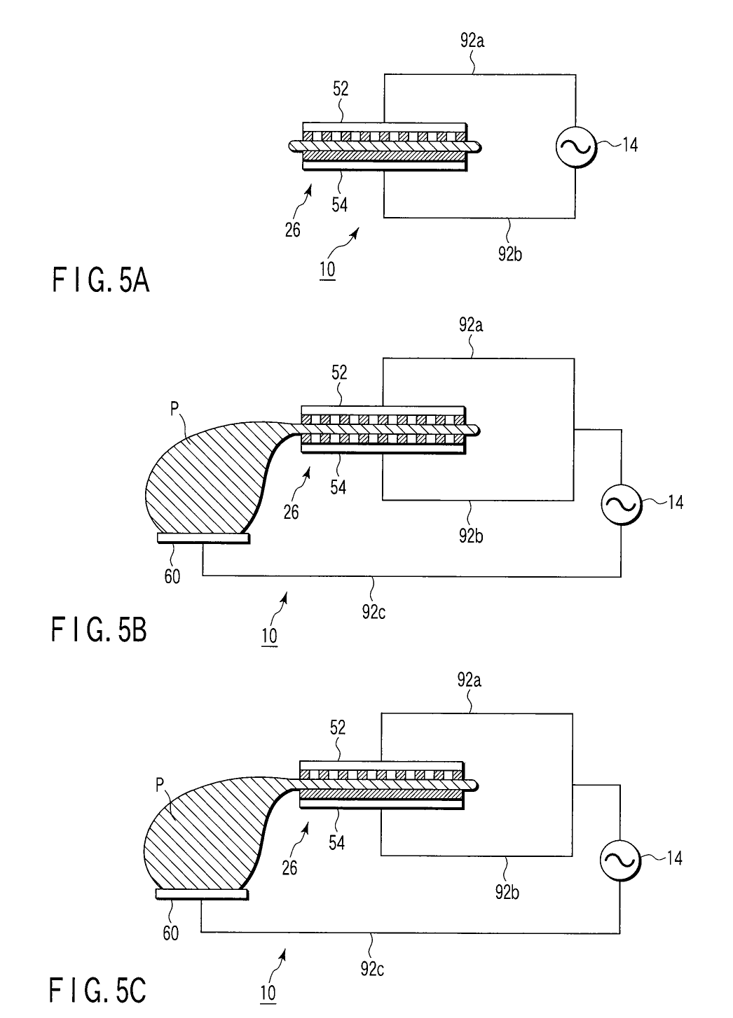

It is to be noted that the holding section 26 when the structure of the main body 62 of the first holding member 52 and the structure of the main body 66 of the second holding member 54 are symmetrical (the same) has been explained in the embodiment. Besides, as shown in FIG. 5A, it is also preferable to adopt the above-explained configuration for the main body 62 of the first holding member 52 and use a second high-frequency electrode 58 like one plane that is entirely exposed on a holding surface on a side close to the first holding member 52 in the main body 66 of the second holding member 54. Even in this case, since the structure of the first high-frequency electrode plate 56 provided on the main body 62 of the first holding member 52 is the same, a treatment can be performed to obtain the same temperature distribution when carrying out the treatment with respect to the living tissue.

Although using the bipolar type electro-surgical device 12 has been explained in the embodiment, using a monopolar type electro-surgical device is also preferable as shown in FIGS. 5B and 5C. In this case, a counter electrode plate 60 is attached to a patient P who is a treatment target. This counter electrode plate 60 is connected with the energy source 14 via the energization line 92c. Further, the first high-frequency electrode plate 56 arranged on the main body 62 of the first holding member 52 and the second high-frequency electrode plate 58 arranged on the main body 66 of the second holding member 54 are in the same potential state where the first and second energization lines 92a and 92b are electrically connected with each other. In this case, since an area of the living tissue that is in contact with the first and second high-frequency electrode plates 56 and 58 is small, current density is high, but current density of the counter electrode plate 60 is low. Therefore, the living tissue held by the holding section 26 generates heat, but heat generation of the living tissue that is in contact with the counter electrode plate 60 is vanishingly small. Therefore, the part held by the holding section 26 alone is heated and, at this time, the living tissue held by the holding section 26 can obtain the further flat temperature distribution in the X axis direction of the main bodies 62 and 66 of the first and second holding members 52 and 54 as explained above.

Furthermore, although not shown, when the monopolar type electro-surgical device is used, arranging the high-frequency electrodes on one of the first holding member 52 and the second holding member 54 alone is also preferable.

Although using the high-frequency electrodes has been explained in this embodiment, ultrasonic transducers or heater elements (not shown) can be used as energy emitting portions in place of adopting the high-frequency electrodes. When using the ultrasonic transducers or the heater elements in this manner, arranging the ultrasonic transducers or the heater elements on at least one of the first and second holding members 52 and 54 enables performing a treatment.

When using, e.g., spot-like ultrasonic transducers in place of the high-frequency electrodes, subjecting these ultrasonic transducers to ultrasonic vibration enables performing a treatment with respect to the living tissue that is in contact with a surface of each ultrasonic transducer like an example where the high-frequency electrodes are used to effect a treatment.

Moreover, when using, e.g., spot-like heater elements in place of the high-frequency electrodes, allowing heat generation from these heater elements enables performing a treatment with respect to the living tissue that is in contact with a surface of each heater element like an example where the high-frequency electrodes are used to effect a treatment.

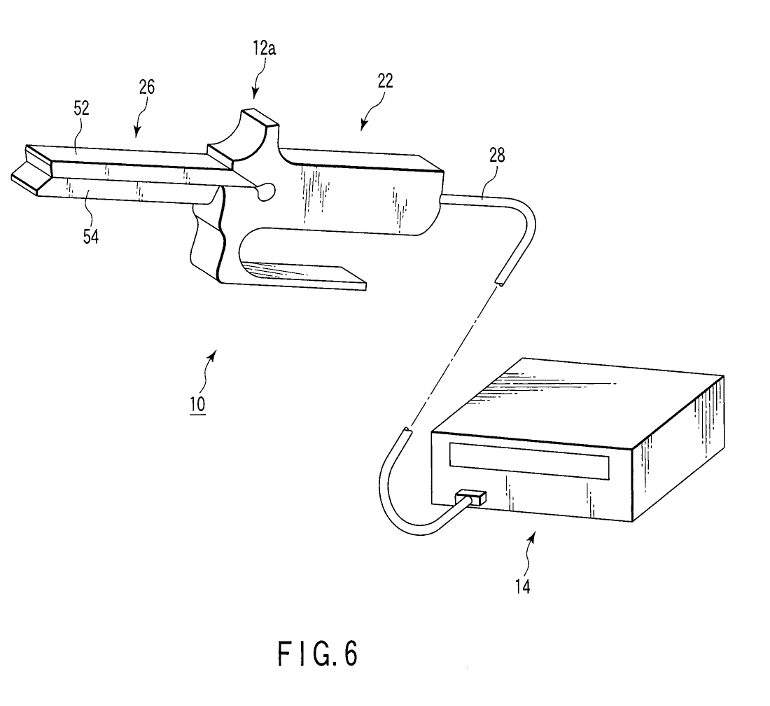

In this embodiment, the linear electro-surgical device 12 for treating the living tissue of the abdominal cavity (in a body) through the abdominal wall has been described as an example. However, for example, as shown in FIG. 6, an open type linear electro-surgical device (a treatment device for curing) 12a may be used which extracts a treatment target tissue out of the body through the abdominal wall to treat the tissue.

The electro-surgical device 12a includes a handle 22 and a holding section 26. That is, unlike the electro-surgical device 12 for treating the tissue through the abdominal wall, the shaft 24 (see FIG. 1A) is omitted. On the other hand, a member having a function similar to that of the shaft 24 is disposed in the handle 22. Therefore, the device can be used in the same manner as in the electro-surgical device 12 described above with reference to FIG. 1A.

### Second Embodiment

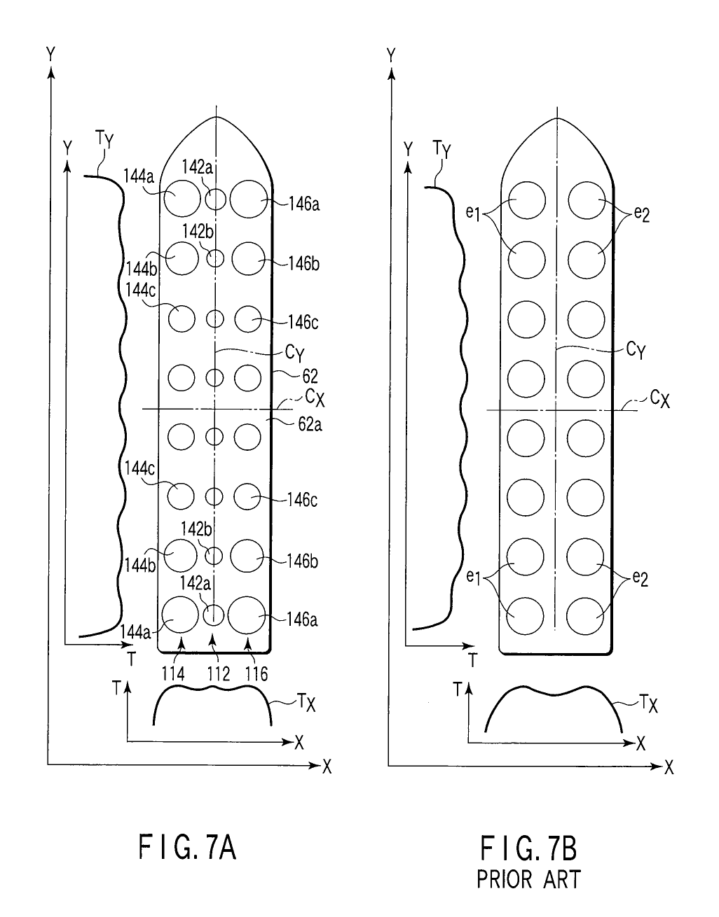

A second embodiment will now be explained with reference to FIGS. 7A and 7B. This embodiment is a modification of the first embodiment, and like reference numerals denote members equal to those explained in the first embodiment, thereby omitting a detailed explanation thereof.

As shown in FIG. 7A, in this embodiment, a first electrode group 112 includes two electrodes 142a and six electrodes 142b. That is, the first electrode group 112 includes the two types of electrodes 142a and 142b. The electrodes 142a are arranged at an upper end and a lower end in FIG. 7A. The electrodes 142b are arranged between the upper end and the lower end in FIG. 7A. An area of each electrode 142a is formed to be larger than an area of each of the other electrodes 142b.

A second electrode group 114 includes two electrodes 144a, two electrodes 144b, and four electrodes 144c. That is, the second electrode group 114 includes the three types of electrodes 144a, 144b, and 144c. The electrodes 144a are arranged at an upper end and a lower end in FIG. 7A. The electrodes 144b are arranged on a lower side adjacent to the upper end and an upper side adjacent to the lower end in FIG. 7A. The electrodes 144c are arranged between the two electrodes 144b. An area of the electrode 144a is formed to be larger than an area of the electrode 144b. And the area of the electrode 144b is formed to be larger than an area of the electrode 144c.

The third electrode group 116 includes three types of electrodes 146a, 146b, and 146c like the second electrode group 114.

It is to be noted that the area of each of the two electrodes 142a provided at the ends of the first electrode group 112 is formed to be smaller than the area of each of the electrodes 144b and 146b in the second electrode group 114 and the third electrode group 116 in FIG. 7A, but it is also preferable for the former area to be equal to or larger than the latter area.

Therefore, the electrodes 142b, 144c, and 146c in two rows on each side, i.e., a total of four rows are symmetrically arranged with a central axis C_{X} perpendicular to the central axis C_{Y} at the center in such a manner that they are close to the central axis C_{X}. The electrodes 142b, 144b, and 146b in each row on each side, i.e., a total of two rows are symmetrically arranged with the central axis C_{X} at the center. Further, the electrodes 142a, 144a, and 146a in each row on each side, i.e., a total of two rows are symmetrically arranged with the central axis C_{X} at the center to sandwich the electrodes 142b, 144c, and 146c in four rows and the electrodes 142b, 144b, and 146b in two rows.

That is, the electrodes 142b, 144c, and 146c in four rows or the electrodes 142b, 144b, and 146b in two rows are arranged at low density in a region of a main body 62 of a first holding member 52 close to the central axis C_{X} (a region close to the central axis). The electrodes 142a, 144a, and 146a in two rows or the electrodes 142b, 144b, and 146b in two rows are arranged in a region of the main body 62 of the first holding member 52 apart from the central axis C_{X} (a region apart from the central axis) at higher density than that in the region close to the central axis. Moreover, an area of each of the electrodes 142b, 144c, and 146c in four rows or the electrodes 142b, 144b, and 146b in two rows in the region close to the central axis that is the region of the main body 62 of the first holding member 52 close to the central axis C_{X} is smaller than an area of each of the electrodes 142a, 144a, and 146a in two rows or the electrodes 142b, 144b, and 146b in tow rows along the X axis direction.

A function of a treatment system 10 according to this embodiment will now be explained.

A living tissue as a treatment target is held between the first holding member 52 and a second holding member 54. In this state, the foot switch or the hand switch is operated. Energy is supplied from an energy source 14 to each of a first high-frequency electrode plate 56 and a second high-frequency electrode plate 58. The living tissue held between the main body 62 of the first holding member 52 and a main body 66 of the second holding member 54 is heated.

It is to be noted that the function in the X axis direction has been explained in the first embodiment, and hence the explanation will be omitted here, and a function in the Y axis direction will be described.

As shown in FIG. 7A, a contact area of each of the electrodes 142a, 144a, and 146a at the ends of the first to third electrode groups 112, 114, and 116 in the Y axis direction with respect to the living tissue is larger than a contact area of each of the electrodes 142b, 144c, and 146c at the central part with respect to the same. Therefore, energy of each of the electrodes 142a, 144a, and 146a in the first to third electrode groups 112, 114, and 116 is larger than energy of each of the electrodes 142b, 144c, and 146c at the central part.

On the other hand, the living tissue that is in contact with the electrodes 142a, 144a, and 146a in the first to third electrode groups 112, 114, and 116 is away from the central axis C_{X} and close to the outside of a holding section 26. Therefore, the living tissue is affected by the outside of the holding section 26 having temperature far lower than that of the living tissue provided between the first holding member 52 and the second holding member 54. However, the living tissue away from the central axis C_{X} of the main body 62 of the first holding member 52 is maintained at high temperature due to functions of the electrodes 142a, 144a, and 146a or the electrodes 14b, 144b, and 146b. Therefore, a heating value near the central axis C_{X} that is given by the electrodes 142b, 144c, and 146c is small, but it further becomes flat.

Thus, temperature distribution T_{Y} of the living tissue when the energy is given to the living tissue from the first high-frequency electrode plate 56 on a surface of the main body 62 of the first holding member 52 in the Y axis direction becomes more flat from a position near the central axis C_{X} to a position corresponding to each edge portion (a distal end and a proximal end of the main body 62 of the first holding member 52) away from the central axis C_{X} of the main body 62 of the first holding member 52. That is, temperature gradient of the living tissue in the holding section 26 along the Y axis direction is reduced as much as possible.

Therefore, the living tissue is uniformly treated in the Y axis direction of the holding section 26. Accordingly, when, e.g., welding the living tissue, the living tissue is uniformly cauterized, thereby obtaining uniform conjugation strength.

Meanwhile, electrodes e₁ and e₂ are arranged in two columns at positions away from the central axis C_{Y} by the same distance in the main body 62 of the first holding member 52 according to the prior art depicted in FIG. 7B. These electrodes e₁ and e₂ are arranged in four rows on each side, i.e., a total of eight rows at positions away from the central axis C_{X} by the same distance. When giving a treatment to a living tissue held by such a first holding member 52, temperature distribution depicted in, e.g., FIG. 7B is demonstrated. In this temperature distribution T_{Y}, a distal end (an upper end) and a proximal end (a lower end) that are apt to be affected by the outside of the holding section 26 show a steep fall as compared with a central part (near the central axis C_{X}), and hence a uniform treatment is hard to be given to the living tissue.

As explained above, according to this embodiment, the following effect can be obtained.

As explained in conjunction with the first embodiment, an arrangement of the electrodes 142a, 144a, and 146a, or the electrodes 142b, 144b, and 146b, or the electrodes 142b, 144c, and 146c along the X axis direction of the main body 62 of the first holding member 52 enables performing a further uniform treatment with respect to the living tissue.

Additionally, as shown in FIG. 7A, the electrodes 142b, 142c, and 146c in four rows, the electrodes 142b, 144b, and 146b in two rows, and the electrodes 142a, 144a, and 146a in two rows are arranged in a direction along which they are away from the central axis C_{X} in a state where these electrodes become symmetrical with respect to the central axis C_{X} in the X axis direction perpendicular to the central axis C_{Y} in the Y axis direction of the main body 62 of the first holding member 52. Further, the contact area of each of the electrodes 142b, 144c, and 146c in four rows with respect to the living tissue is set to be smaller than the contact area of each of the electrodes 142b, 144b, and 146b in two rows arranged on the outer side. The contact area of each of the electrodes 142b, 144b, and 146b in two rows with respect to the living tissue is set to be smaller than the contact area of each of the electrodes 142a, 144a, and 146a in two rows arranged on the outer side. That is, an amount of energy supplied to the living tissue from each of the electrodes 142b, 144c, and 146c in four rows is set to be smaller than an amount of energy supplied to the living tissue from each of the electrodes 142b, 144b, and 146b in two rows. The amount of energy supplied to the living tissue from each of the electrodes 142b, 144b, and 146b in two rows is set to be smaller than an amount of energy supplied to the living tissue from each of the electrodes 142a, 144a, and 146a in two rows.

Then, the temperature distribution T_{Y} in the Y axis direction that is supplied to the living tissue from the main body 62 of the first holding member 52 depicted in FIG. 7A can be uniformed from a position corresponding to the distal end to a position corresponding to the proximal end of the main body 62 of the first holding member 52 as compared with the temperature distribution T_{Y} of the prior art depicted in FIG. 7B. That is, the temperature gradient of the temperature distribution T_{Y} in the Y axis direction supplied to the living tissue from the main body 62 of the first holding member 52 depicted in FIG. 7A can be further flattened as compared with the temperature gradient of the temperature distribution T_{Y} of the prior art shown in FIG. 7B. Then, an arrangement of the electrodes in the Y axis direction of the main body 62 of the first holding member 52 enables performing a further uniform treatment, e.g., weld or cautery with respect to the living tissue.

Therefore, according to this embodiment, both the temperature distribution T_{X} and T_{Y} supplied to the living tissue can be further uniformed in both the X axis direction and the Y axis direction of the main body 62 of the first holding member 52.

### Third Embodiment

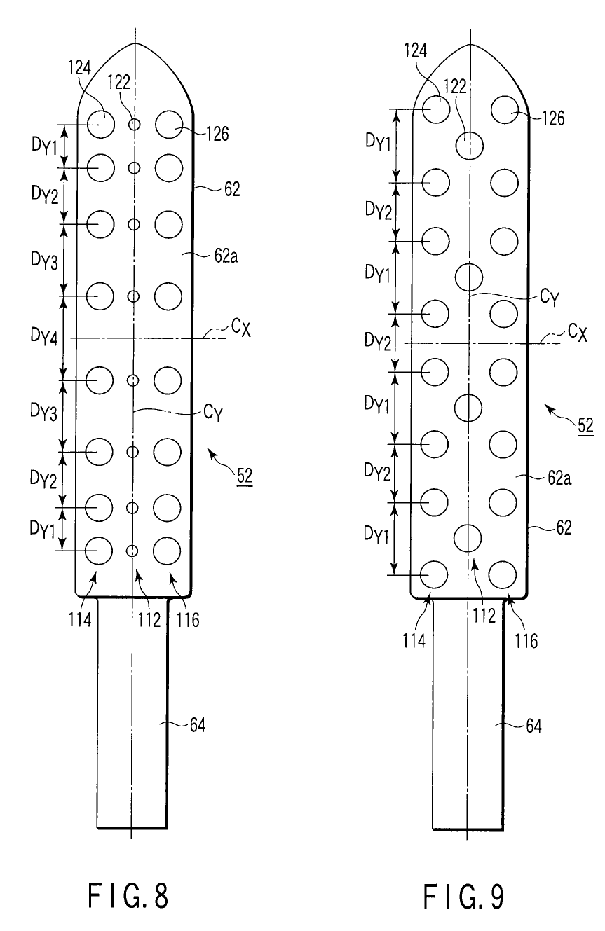

A third embodiment will now be explained with reference to FIG. 8. This embodiment is a modification of the first and second embodiments, and like reference numerals denote members equal to those explained in the first and second embodiments, thereby omitting a detailed explanation.

Respective electrodes 122 in a first electrode group 112 have the same area, as shown FIG. 8. Respective electrodes 124 and 126 in second and third electrode groups 114 and 116 likewise have the same area.

Therefore, as explained in the first embodiment, an arrangement of the electrodes 122, 124, and 126 in the X axis direction of a main body 62 of a first holding member 52 enables performing a further uniform treatment, e.g., weld or cautery with respect to a living tissue.

There are four types of distances D_{Y1}, D_{Y2}, D_{Y3}, and D_{Y4} between centers of the electrodes 124 in the second electrode group 114. The distance D_{Y1} is an intercentral distance between the electrode 124 on the outermost end side in the Y axis direction and the next electrode 124 on the inner side along the Y axis direction. Furthermore, the distance D_{Y2} is an intercentral distance between the electrode 124 one position down on the inner side from the end in the Y axis direction and the next electrode 124 two positions down on the inner side from the end. Moreover, the distance D_{Y3} is an intercentral distance between the electrode 124 two positions down on the inner side from the end in the Y axis direction and the next electrode 124 on three positions down on the inner side. Additionally, the distance D_{Y4} is an intercentral distance between the electrodes 124 that are closest to a central axis C_{X} in the X axis direction.

At this time, the distance D_{Y1} is shortest, the distance D_{Y2} is second-shortest, the distance D_{Y3} is third-shortest, and the distance D_{Y4} is longest. Therefore, density of the electrodes 124 is high on the end side in the Y axis direction, and it is low on the central side in the same direction.

These relationships are applied to not only the second electrode group 114 but also the first electrode group 112 and the third electrode group 116.

Here, the living tissue on the end side is apt to be affected by external temperature of a holding section 26 in the Y axis direction like the X axis direction as compared with the central side. Therefore, as explained in conjunction with the second embodiment, an arrangement of the electrodes while changing the gaps in the Y axis direction in the main body 62 of the first holding member enables performing a further uniform treatment, e.g., weld or cautery with respect to the living tissue. That is, when the density of the electrodes is changed to be high on the end side and low on the central side along the Y axis direction, temperature gradient of temperature distribution T_{Y} in the Y axis direction given to the living tissue from the main body 62 of the first holding member 52 can be further flattened.

Therefore, according to this embodiment, in both the X axis direction and the Y axis direction of the main body 62 of the first holding member 52, both the temperature distribution T_{X} and T_{Y} given to the living tissue can be further uniformed.

### Fourth Embodiment

A fourth embodiment will now be explained with reference to FIG. 9. This embodiment is a modification of the first and second embodiments, and like reference numerals denote members equal to those in the first and second embodiments, thereby omitting a detailed explanation thereof.

As shown in FIG. 9, respective electrodes 122, 124, and 126 in first to third electrode groups 112, 114, and 116 have the same area.

There are two types of distances D_{Y1} and D_{Y2} between centers of the electrodes 124 in the second electrode group 114 that are adjacent to each other in the Y axis direction. The distance D_{Y1} is an intercentral distance of the respective electrodes 124 on the outermost end side in the Y axis direction. Further, the distance D_{Y2} is an intercentral distance between the respective electrodes 124 one position down on the inner side from the end in the Y axis direction. The distances D_{Y1} and D_{Y2} are alternately repeated between the centers of the electrodes 124 adjacent to each other in the second electrode group 114. Such a relationship is likewise applied to the respective electrodes 126 in the third electrode group 116.

On the other hand, the first electrode group 112 includes the four electrodes 122 whose number is fewer than those explained in the first and second embodiments. Distances between centers of the respective electrodes 122 are equal to each other. Furthermore, the respective electrodes 122 in the first electrode group 112 are arranged with a position at which diagonal lines connecting the centers of the electrodes 124 and 126 arranged with the distance D_{Y1} cross each other being determined as the center. That is, the electrodes 122, 124, and 126 are arranged on a holding surface 62a of the main body 62 of the first holding member 52 like a five-spot of a dice.

As explained above, parts that look like the five-spot of the dice are provided at four positions at equal intervals from a distal end side toward a proximal end side of the holding surface 62a of the first holding member 62 in the Y axis direction. That is, they are symmetrically provided at two position on each side with respect to the central axis C_{X}. In particular, the parts that look like the five-spot of the dice are also provided at both the distal end and the proximal end of the holding surface 62a of the first holding member 62 in the Y axis direction. Therefore, on the holding surface 62a of the main body 62 of the first holding member 52, density of the electrodes 122, 124, and 126 on the distal end side and the proximal end side in the Y axis direction is high and density of the electrodes 122, 124, and 126 on the central side close to the central axis C_{X} is low as a whole.

Therefore, as explained in conjunction with the second embodiment, an arrangement of the electrodes in the Y axis direction of the main body 62 of the first holding member 52 enables forming a uniform treatment, e.g., weld or cautery with respect to the living tissue.

Moreover, each electrode 122 in the first electrode group 112 according to this embodiment has the same area as that of each electrode 124 or 126 in the second or third electrode group 114 or 116. Therefore, an amount of energy supplied to the living tissue from each electrode 122 in the first electrode is larger than that explained in the first embodiment.

Therefore, as explained in the first embodiment, an arrangement of the electrodes 122, 124, and 126 in the X axis direction of the main body 62 of the first holding member 52 enables performing a further uniform treatment, e.g., weld or cautery with respect to the living tissue.

Therefore, according to this embodiment, in both the X axis direction and the Y axis direction of the main body 62 of the first holding member 52, both the temperature distribution T_{X} and T_{Y} given to the living tissue can be further uniformed.

### Fifth Embodiment

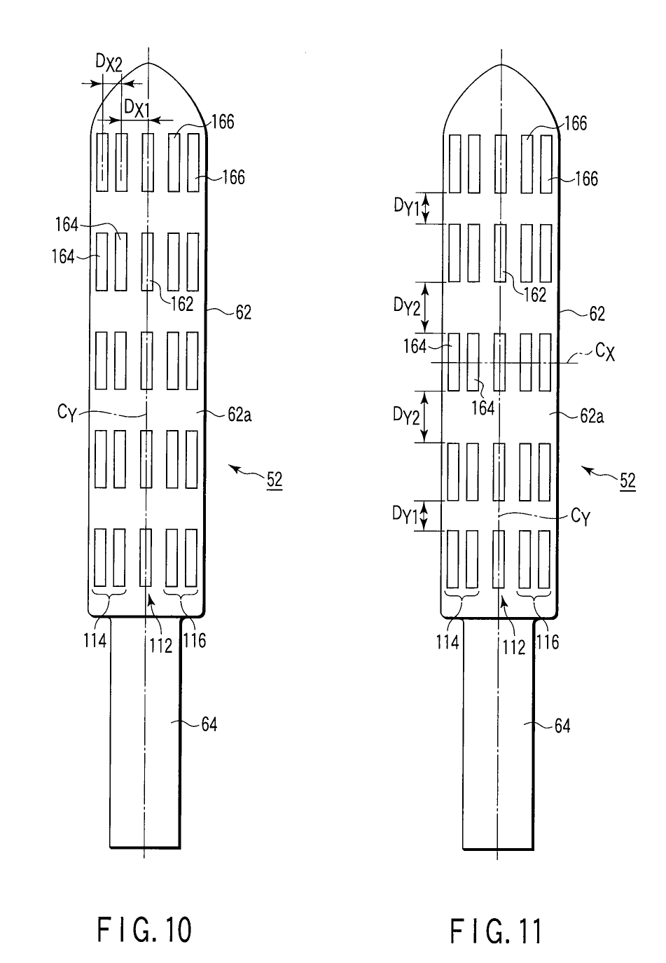

A fifth embodiment will now be explained with reference to FIG. 10. This embodiment is a modification of the first embodiment, and like reference numerals denote members equal to those explained in the first embodiment, thereby omitting a detailed explanation.

As shown in FIG. 10, in this embodiment, a first electrode group 112 includes five rectangular electrodes 162 in one column. A longitudinal direction of each electrode 162 is a Y axis direction. The respective electrodes 162 are arranged in the Y axis direction at equal intervals.

Each of second and third electrode groups 114 and 116 includes ten rectangular electrodes 164 or 166. The electrodes 164 or 166 in the second or third electrode group 114 or 116 are arranged in two columns in an X direction and five rows in the Y axis direction. That is, as to the electrodes 164 or 166 in two columns, the five electrodes 164 or 166 are arranged in each column at equal intervals in the Y axis direction. Intervals of the electrodes 164 or 166 in the second or third electrode group 114 or 116 along the X axis direction are equal to each other. A longitudinal direction of the respective electrodes 164 or 166 is the Y axis direction.

It is to be noted that an area of each electrode 162 in the first electrode group 112 is substantially equal to areas of the respective electrodes 164 and 166 in the second and third electrode groups 114 and 116.

Moreover, a distance D_{X1} between a central axis of the electrode 162 in the first electrode group 112 and a central axis of the electrode 164 in the second electrode group 114 that is provided in a column close to the first electrode group 112 is formed to be longer than a distance D_{X2} between central axes of the electrodes 164 in the second electrode group 114 that are provided in two columns.

This is also applied to a relationship between the first electrode group 112 and the third electrode group 116. Therefore, density of the first electrode group 112 is lower than those of the second and third electrode groups 114 and 116.

A function of a treatment system 10 according to this embodiment will now be explained.

As shown in FIG. 10, although the respective electrodes 162 in the first electrode group 112 are provided in one column, the respective electrodes 164 or 166 in the second electrode group 114 or the third electrode group 116 are provided in two columns. Therefore, a contact area of each electrode 162 in the first electrode group 112 with respect to a living tissue is smaller than a contact area of each electrode 164 or 166 in the second electrode group 114 or the third electrode group 116 with respect to the same. Accordingly, total energy of the electrodes 164 or 166 in the second electrode group 114 or the third electrode group 116 that are provided in two columns respectively is larger than energy of the electrodes 162 in the first electrode group 112 that are provided in one column.

On the other hand, the living tissue that is in contact with the second electrode group 114 or the third electrode group 116 is away from the central axis C_{Y} and close to the outside of a holding section 26. Hence the living tissue is affected by the outside of the holding section 26 having temperature far lower than that of the living tissue provided between the first holding member 52 and a second holding member 54. However, the living tissue near the central axis C_{Y} of the main body 62 of the first holding member 52 is maintained at high temperature due to functions of the second and third electrode groups 114 and 116. Therefore, even though a heating value near the central axis C_{Y} that is given by the first electrode group 112 is small, it is further flattened.

Therefore, temperature distribution T_{X} of the living tissue held by the holding section 26 when energy on a surface of the main body 62 of the first holding member 52 in the X axis direction given to the living tissue is approximated to a further flat state down to a position corresponding to an edge portion away from the central axis C_{Y} of the main body 62 of the first holding member 52. That is, temperature gradient of the living tissue in the X axis direction in the holding section 26 is reduced as much as possible.

Therefore, the living tissue is uniformly treated in the X axis direction of the holding section 26. Accordingly, when, e.g., welding the living tissue, the living tissue is uniformly cauterized, thereby obtaining uniform conjugation strength.

Therefore, an arrangement of the electrodes 162 in the first electrode group 112 that are provided in one column and the electrodes 164 or 166 in the second or third electrode group 114 and 116 that are provided in two columns in the X axis direction of the main body 62 of the first holding member 52 enables performing a further uniform treatment, e.g., weld or cautery with respect to the living tissue.

It is to be noted that each of the electrodes 162, 164, and 166 in the first to third electrode groups 112, 114, and 116 has a rectangular shape in the explanation of this embodiment, but various shapes, e.g., an elliptic shape can be allowed.

Although the electrodes 164 or 166 in the second or third electrode group 114 or 116 are provided in two columns in the explanation of this embodiment, forming the two electrodes 164 or 166 in the second or third electrode group 114 or 116 that are adjacent to each other in the X axis direction as one electrode is also preferable.

### Sixth Embodiment

A sixth embodiment will now be explained with reference to FIG. 11. This embodiment is a modification of the first, third, and fifth embodiments, and like reference numerals denote members equal to those explained in conjunction with the first, third, and fifth embodiments, thereby omitting a detailed explanation.

As shown in FIG. 11, each of electrodes 162, 164, and 166 in first to third electrode groups 112, 114, and 116 according to this embodiment has a rectangular shape as explained in conjunction with the fifth embodiment.

There are two types of distances D_{Y1} and D_{Y2} between ends of the respective electrodes 162, 164, and 166 in the first to third electrode groups 112, 114, and 116. The distance D_{Y1} is a distance between the electrode 162, 164, or 166 provided at the outermost end and the next electrode 162, 164, or 166 on the inner side from the end in a Y axis direction. The distance D_{Y2} is a distance between the electrode 162, 164, or 166 one position down on the inner side from the end in the Y axis direction and the electrode 162, 164, or 166 two positions down from the end in the Y axis direction. The distance D_{Y1} is shorter than the distance D_{Y2}. Therefore, density of the electrodes 162, 164, and 166 are high one the end side and the density of the same is low than the end side on the central side in the Y axis direction.

Therefore, a living tissue is uniformly treated in the X axis direction of a holding section 26. Accordingly, when, e.g., welding the living tissue, the living tissue is uniformly cauterized, thereby obtaining uniform conjugation strength.

Here, the living tissue is apt to be affected by external temperature of the holding section 26 on the end side in the Y axis direction as compared with the central side like the X axis direction. Therefore, an arrangement of the electrodes in the Y axis direction of the main body 62 of the first holding member 52 enables performing a uniform treatment, e.g., weld or cautery with respect to the living tissue.

Thus, according to this embodiment, both temperature distribution T_{X} and T_{Y} that are given to the living tissue can be further uniformed in both the X axis direction and the Y axis direction of the main body 62 of the first holding member 52.

Therefore, an arrangement of the electrodes in the Y axis direction of the main body 62 of the first holding member 52 enables performing a further uniform treatment, e.g., weld or cautery with respect to the living tissue.

### Seventh Embodiment

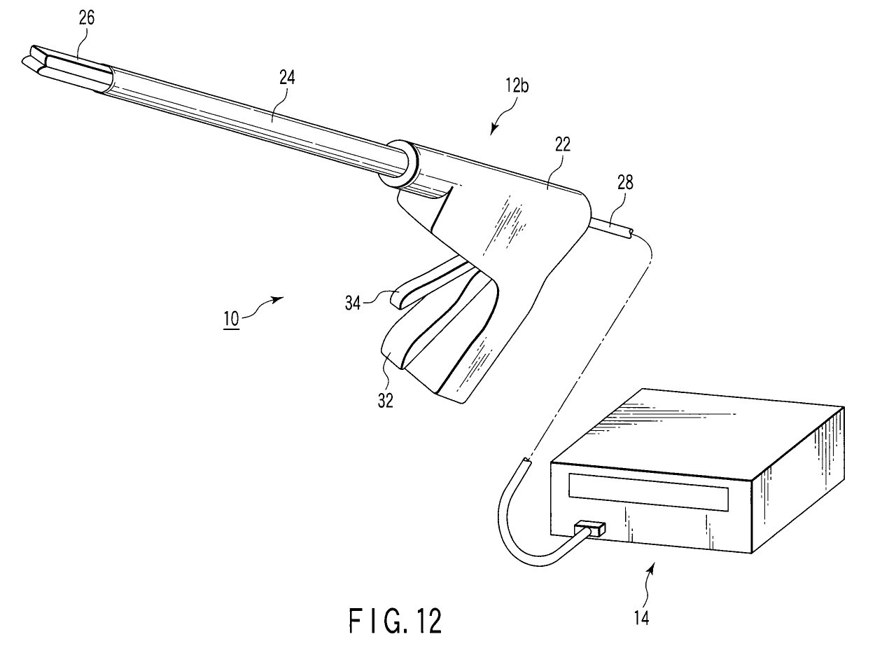

A seventh embodiment will now be explained with reference to FIGS. 12 to 14. This embodiment is a modification of the first to sixth embodiments, and like reference numerals denote members equal to those explained in the first to sixth embodiments or members having the same functions, thereby omitting a detailed explanation.

As shown in FIG. 12, a handle 22 of an electro-surgical device (a treatment device for curing) 12b according to this embodiment is provided with a cutter driving knob 34 disposed along a holding section opening/closing knob 32.

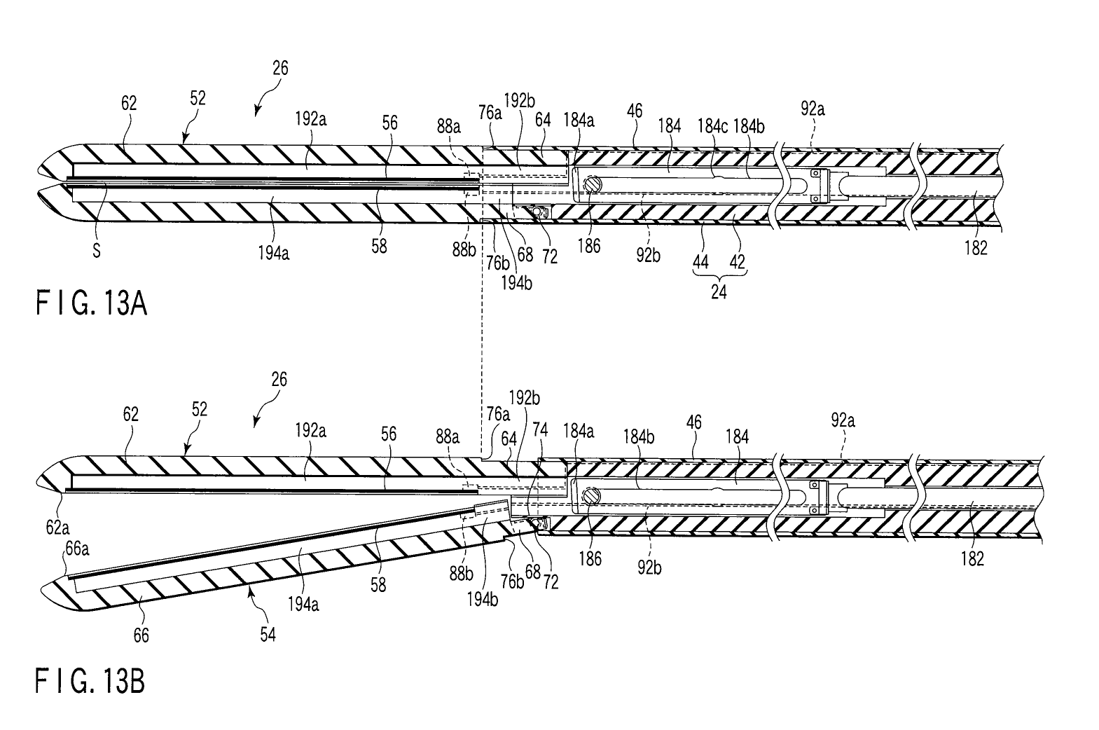

As shown in FIGS. 13A and 13B, a driving rod 182 is movably disposed along an axial direction of a cylindrical member 42 in the cylindrical member of a shaft 24. A distal end of the driving rod 182 is provided with a thin-plate-like cutter 184. Therefore, when the cutter driving knob 34 is operated, the cutter (an auxiliary curative device) 184 moves via the driving rod 182.

As shown in FIGS. 13A and 13B, a distal end of the cutter 184 is provided with a blade 184a, and the distal end of the driving rod 182 is fixed to a proximal end of the cutter 184. A longitudinal groove 184b is formed between the distal end and the proximal end of the cutter 184. Engagement portions 184c which engage with a movement regulation pin 186 are formed on one end of the longitudinal groove 184b, the other end and between one end and the other end. In the longitudinal groove 184b, the movement regulation pin 186 extending in a direction crossing the axial direction of the shaft 24 at right angles is fixed to the cylindrical member 42 of the shaft 24. Therefore, the longitudinal groove 184b of the cutter 184 moves along the movement regulation pin 186. In this case, the cutter 184 linearly moves. At this time, the cutter 184 is disposed along cutter guide grooves 192a, 192b, 194a and 194b of a first holding member 52 and a second holding member 54.

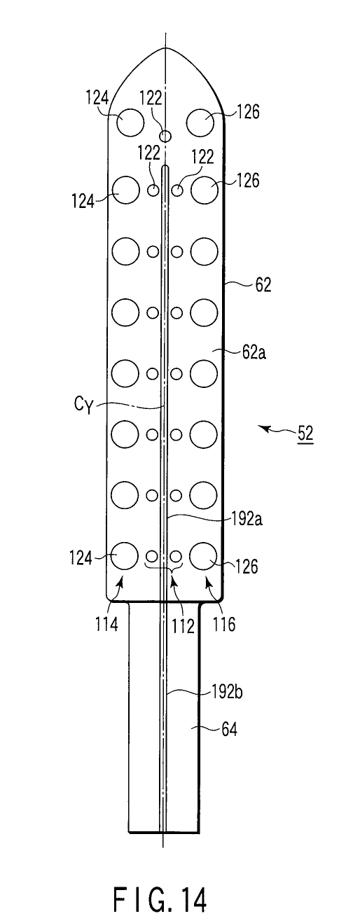

As shown in FIG. 14, the cutter guide grooves 192a and 192b are formed on a central axis C_{Y} of the first holding member 52 on a side close to the second holding member 54. A distal end (an upper end) of the cutter guide groove 192a of a main body 62 of the first holding member 52 in FIG. 14 is present between, e.g., a distal end (an upper end) and a proximal end (a lower end) of the main body 62.

An electrode 122 at the uppermost end in a first electrode group 112 is arranged on the distal end side apart from the upper end of the cutter guide groove 192a. The remaining electrodes 122 in the first electrode groove 122 are symmetrically arranged with a central axis of the main body 62 having the cutter guide groove 192a provided therein at the center along a Y axis direction at equal intervals. Therefore, the remaining electrodes 122 in the first electrode group 112 are arranged to face the cutter guide groove 192a formed in the main body 62. In particular, an area of each electrode 122 in the first electrode group 112 is smaller than those of respective electrodes 124 and 126 in second and third electrode groups 114 and 116.

A function of a treatment system 10 according to this embodiment will now be explained.

As explained in conjunction with the first embodiment, the temperature distribution T_{X} (see FIG. 4A) of a living tissue held by the holding section 26 when energy from the surface (the holding surface) of the main body 62 of the first holding member 52 in the X axis direction applied to the living tissue is approximated to a further flat state down to a position corresponding to an edge portion away from the central axis C_{Y} of the main body 62 of the first holding member 52. That is, the temperature gradient of the living tissue in the X axis direction of the holding section 26 is reduced as much as possible.

Therefore, the living tissue is uniformly treated in the X axis direction of the holding section 26. Therefore, when, e.g., welding the living tissue, the living tissue is uniformly cauterized, thereby obtaining uniform conjugation strength.

Further, after the living tissue is subjected to a heat treatment, the cutter driving knob 34 of the handle 22 is operated. Then, the cutter 174 moves toward the distal ends of the first holding member 52 and the second holding member 54. Since the cutter 174 has the blade 174a at the distal end thereof, thereby cutting the treated living tissue.

Therefore, as explained in conjunction with the first embodiment, an arrangement of the electrodes 122, 124, and 126 in the X axis direction of the main body 62 of the first holding member 52 enables performing a further uniform treatment with respect to the living tissue.

It is to be noted that the electrode 122 provided at the uppermost end in the first electrode group 112 in FIG. 14 is arranged on a lower side than the electrodes 124 and 126 provided at the uppermost ends in the second electrode group 114 and the third electrode group 116, but arranging these electrodes in parallel with each other along the X axis direction is also preferable.

Furthermore, the main bodies 62 and 66 of the first and second holding members 52 and 54 having such shapes as explained in the first to sixth embodiments are also allowed. In such a case, arranging the respective electrodes in the first electrode group 112 in, e.g., two columns as shown in FIG. 14 can suffice. It is preferable for each electrode in the first electrode group 112 to have an area that is approximately ½ of an area of each of the electrodes 122, 142a, 142b, and 162 in the first electrode group 112 explained in the first to sixth embodiments.

### Eighth Embodiment

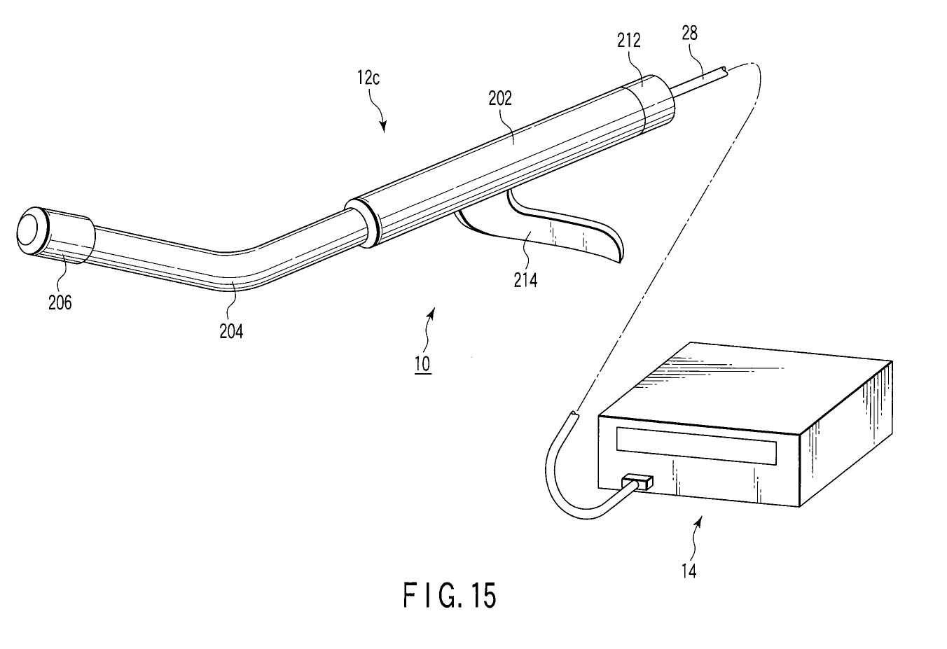

An eighth embodiment will now be explained with reference to FIGS. 15 to 18B.

Here, as an example of an energy treatment device, a circular type bipolar electro-surgical device (a treatment device for curing) 12c will be described which performs a treatment, for example, through an abdominal wall or outside the abdominal wall.

As shown in FIG. 15, the electro-surgical device 12c includes a handle 202, a shaft 204 and an openable/closeable holding section 206. The handle 202 is connected with an energy source 14 via a cable 28.

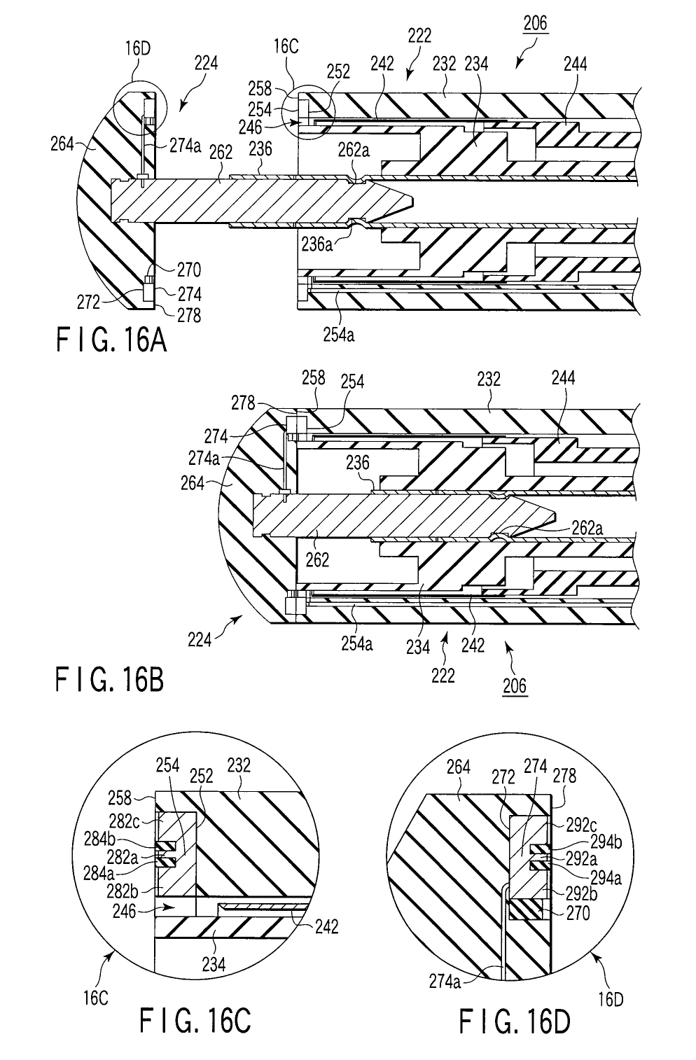

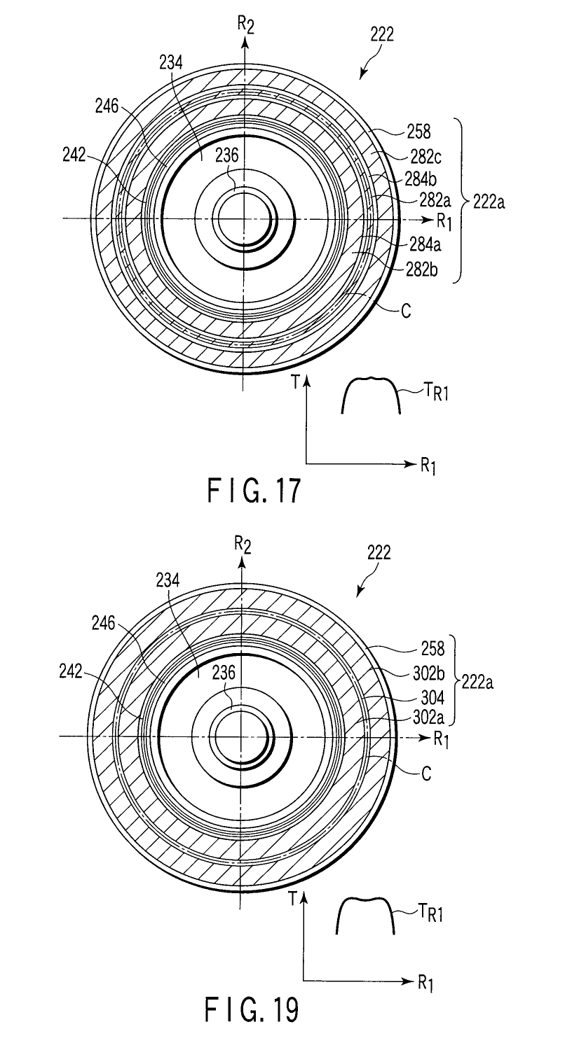

The handle 202 is provided with a holding section opening/closing knob 212 and a cutter driving lever 214. The holding section opening/closing knob 212 is rotatable with respect to the handle 202. When the holding section opening/closing knob 212 is rotated, for example, clockwise with respect to the handle 202, a detachable side holding portion 224 of the holding section 206 described later comes away from a main body side holding portion 222 (see FIG. 16A). When the knob is rotated counterclockwise, the detachable side holding portion 224 comes close to the main body side holding portion 222 (see FIG. 16B).

The shaft 204 is formed into a cylindrical shape. This shaft 204 is appropriately curved in consideration of an insertion property into a living tissue. Needless to say, the shaft 204 may linearly be formed.

A distal end of the shaft 204 is provided with the holding section 206. As shown in FIGS. 16A and 16B, the holding section 206 includes the main body side holding portion (a first holding member) 222 formed at the distal end of the shaft 204, and the detachable side holding portion (a second holding member) 224 detachably attached to the main body side holding portion 222.

The main body side holding portion 222 includes a cylindrical member 232, a frame 234 and an electric conductive pipe 236. The cylindrical member 232 and the frame 234 have an insulating property. The cylindrical member 232 is connected with the distal end of the shaft 204. The frame 234 is fixed to the cylindrical member 232.

A central axis of the frame 234 is opened. The opened central axis of the frame 234 is provided with the electric conductive pipe 236 which is movable in a predetermined region along the central axis of the frame 234. When the holding section opening/closing knob 212 is rotated, as shown in FIGS. 16A and 16B, the electric conductive pipe 236 is movable in a predetermined region owing to, for example, a function of a ball screw (not shown). The electric conductive pipe 236 is provided with a protrusion 236a which protrudes inwards in a diametric direction so that a connecting portion 262a of an electric conductive shaft 262 described later disengageably engages with the protrusion.

As shown in FIGS. 16A and 16B, a space is formed between the cylindrical member 232 and the frame 234. A cylindrical cutter 242 is disposed in the space between the cylindrical member 232 and the frame 234. A proximal end of the cutter 242 is connected to a distal end of a pusher 244 for the cutter disposed in the shaft 204. The cutter 242 is fixed to an outer peripheral surface of the pusher 244 for the cutter. Although not shown, a proximal end of the pusher 244 for the cutter is connected to the cutter driving lever 214 of the handle 202. Therefore, when the cutter driving lever 214 of the handle 202 is operated, the cutter 242 moves via the pusher 244 for the cutter 242.

As shown in FIGS. 16A and 16C, a distal end of the cylindrical member 232 is provided with an annular electrode arrangement portion 252. A first high-frequency electrode 254 is disposed as an output portion or an energy applying portion at the electrode arrangement portion 252. A distal end of a first conducting line 254a is fixed to the first high-frequency electrode ring 254. The first conducting line 254a is connected to the cable 28 via the main body side holding portion 222, the shaft 204 and the handle 202.

As shown in FIGS. 16A, 16C, 17, and 18A, an edge portion 258 is formed on an outer side of the first high-frequency electrode ring 254.

As shown in FIGS. 16C, 17, and 18A, the first high-frequency electrode ring 254 includes a first annular electrode 282a, a second annular electrode 282b, and a third annular electrode 282c. Of these electrodes, the first annular electrode 282a is formed near a central line C between an inner circumference and an outer circumference of the first high-frequency electrode ring 254 (a region near a central axis as a first region). The second annular electrode 282b is formed on an inner side of the first annular electrode 282a. The third annular electrode 282c is formed on an outer side of the first annular electrode 282a (a region away from the central axis as the second and/or third region). A width of the first annular ring 282a in a radial direction (an R₁ direction) is smaller than widths of the second and third annular electrodes 282b and 282c. The widths of the second and third annular electrodes 282b and 282c are substantially equal to each other.

Further, an annular first insulating member 284a is arranged between the first annular electrode 282a and the second annular electrode 282b. An annular second insulating member 284b is arranged between the first annular electrode 282a and the third annular electrode 282c.

The first to third annular electrodes 282a, 282b, and 282c of the first high-frequency electrode ring 254, the first and second insulating members 284a and 284b, and the edge portion 258 of a holding section 222 on a main body side are a holding surface 222a of the main body side holding section 222 with respect to a living tissue.

On the other hand, as shown in FIGS. 16A and 16B, the detachable side holding portion 224 includes an energization shaft 262 having a connecting portion 262a, and a head portion 264. The energization shaft 262 has a circular cross section, one end formed into a tapered shape, and the other end being fixed to the head portion 264. The connecting portion 262a is formed into a concave groove shape allowing engagement with a protrusion 236a of the energization pipe 236. An outer surface of the energization shaft 262 except the connecting portion 262a is insulated by using, e.g., a coating.

As shown in FIGS. 16A, 16B, 16D, and 17, a cutter receiving portion 270 having an annular shape is provided in the head portion 264. An annular electrode arrangement portion 272 is formed on an outer side of this cutter receiving portion 270. A second high-frequency electrode ring 274 as an output member or an energy applying portion is provided in the electrode arrangement portion 272. One end of a second energization line 274a is fixed to this second high-frequency electrode ring 274. The other end of the second energization line 274a is electrically connected with the energization shaft 262. A contact surface of an edge portion 278 is formed on an outer side of this second high-frequency electrode ring 274.

It is to be noted that the energization pipe 236 is connected with the cable 28 through the shaft 204 and the handle 202. Therefore, when the connecting portion 262a of the energization shaft 262 of the detachable side holding portion 224 is engaged with the protrusion 236a of the energization pipe 236, the second high-frequency electrode ring 274 is electrically connected with the energization pipe 236.

As shown in FIGS. 16D, 17, and 18A, the second high-frequency electrode ring 274 includes a first annular electrode 292a, a second annular electrode 292b, and a third annular electrode 292c. Of these electrodes, the first annular electrode 292a is formed near a central line C between an inner circumference and an outer circumference of the second high-frequency electrode ring 274. The second annular electrode 292b is formed on the inner side of the first annular electrode 292a. The third annular electrode 292c is formed on the outer side of the first annular electrode 292a. A width of this first annular electrode 292a in a radial direction (an R₁ direction) is smaller than widths of the second and third annular electrodes 292b and 292c. The widths of the second and third annular electrodes 292b and 292c are substantially equal to each other.

Furthermore, an annular first insulating member 294a is arranged between the first annular electrode 292a and the second annular electrode 292b. An annular second insulating member 294b is arranged between the first annular electrode 292a and the third annular electrode 292c.

A function of a treatment system 10 according to this embodiment will now be explained.

As shown in FIG. 16B, the holding section 206 and the shaft 204 of the electro-surgical device 12c are inserted into, e.g., an abdominal cavity through an abdominal wall in a state where the main body side holding section 222 is closed with respect to the detachable side holding portion 224. The main body side holding portion 222 and the detachable side holding portion 224 of the electro-surgical device 12c is opposed to the living tissue to be treated.

The holding section opening/closing knob 212 of the handle 202 is operated to grasp the living tissue as a treatment target by the main body side holding section 222 and the detachable side holding portion 224. At this time, the holding section opening/closing knob 212 is rotated, e.g., clockwise with respect to the handle 202. Then, as shown in FIG. 16A, the energization pipe 236 is moved to the distal end side with respect to the frame 234 of the shaft 204. Therefore, the space between the main body side holding section 222 and the detachable side holding portion 224 is opened, thereby detaching the detachable side holding portion 224 from the main body side holding section 222.

Moreover, the living tissue as a treatment target is arranged between the first high-frequency electrode ring 254 of the main body side holding section 222 and the second high-frequency electrode ring 274 of the detachable side holding portion 224. The energization shaft 262 of the detachable side holding portion 224 is inserted into the energization pipe 236 of the main body side holding section 222. In this state, the holding section opening/closing knob 212 of the handle 202 is rotated, e.g., counterclockwise. Therefore, the detachable side holding portion 224 is closed with respect to the main body side holding section 222. In this manner, the living tissue as a treatment target is held between the main body side holding section 222 and the detachable side holding portion 224.

In this state, the foot switch or the hand switch is operated, and energy is thereby supplied to the first high-frequency electrode ring 254 and the second high-frequency electrode ring 274 from an energy source 14 via the cable 28. The first to third annular electrodes 282a, 282b, and 282c of the first high-frequency electrode ring 254 apply a high-frequency current to a space between themselves and the first to third annular electrodes 292a, 292b, and 292c of the second high-frequency electrode ring 274 via the living tissue. Therefore, the living tissue between the main body side holding section 222 and the detachable side holding portion 224 is heated.

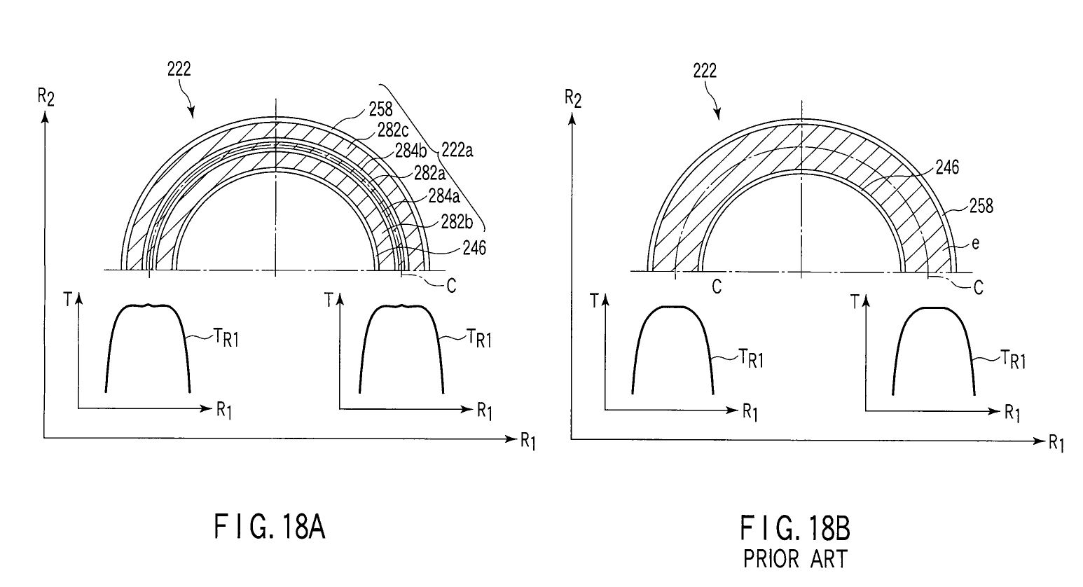

At this time, as shown in FIGS. 17 and 18A, a contact area or a width in a radial direction (an R₁ axis direction in FIGS. 17 and 18A) of the first annular electrode 282a near the central line C with respect to the living tissue is smaller than that of the second annular electrode 282b or the third annular electrode 282c away from the central line C. Therefore, energies applied to the living tissue from the second annular electrode 282b and the third annular electrode 282c are larger than energy applied to the living tissue from the first annular electrode 282a.

On the other hand, the living tissue that is in contact with the second annular electrode 282b or the third annular electrode 282c is away from the central line C and close to the outside of the holding section 206. Hence, the living tissue is affected by the outside of the holding section 206 having temperature far lower than that of the living tissue present between the main body side holding section 222 and the detachable side holding portion 224. However, the living tissue near the central line C of the main body side holding section 222 is maintained at high temperature due to a function of the second annular electrode 282b or the third annular electrode 282c. Therefore, even though a heating value near the central line C that is given by the first annular electrode 282a is small, it is further flattened.

Therefore, temperature distribution T_{R1} of the living tissue held by the holding section 206 when energy from a surface (a holding surface) of the main body side holding section 222 in the holding section 206 in the R₁ axis direction supplied to the living tissue is approximated to a further flat state down to a position corresponding to an edge portion away from the central line C of the main body side holding section 222. That is, temperature gradient of the living tissue in the R₁ axis direction in the holding section 26 is reduced as much as possible.

Therefore, the living tissue is uniformly treated in the R₁ axis direction of the holding section 206. Accordingly, when, e.g., welding the living tissue, the living tissue is uniformly cauterized, thereby obtaining uniform conjugation strength.

Additionally, when the cutter driving lever 214 of the handle 202 is operated, a cutter 242 protrudes from a space 246 of the main body side holding section 222 and moves toward a cutter receiving portion 270 of the detachable side holding portion 224. Since the cutter 242 has a blade at a distal end thereof, the treated living tissue is cut into a circular shape.

Meanwhile, one annular electrode e is arranged along the central line C on a holding section 222 on a main side according to the prior art depicted in FIG. 18B. When providing a treatment with respect to a living tissue held by such a holding section 222 on the main side, temperature distribution T_{R1} depicted in FIG. 18B is demonstrated, for example. This temperature distribution T_{R1} is flat at the center, but temperature of the living tissue present at a position corresponding to an inner circumference and an edge portion of an outer circumference is precipitously reduced due to, e.g., an influence of the outside of the holding section 206. Therefore, in the temperature distribution T_{R1} in the R₁ axis direction with respect to a width of the annular electrode e, a drop of the temperature at a position corresponding to the edge portion is large. That is, when using the main body side holding section 222 according to the prior art depicted in FIG. 18B, performing a uniform treatment with respect to the living tissue is difficult.

As explained above, according to this embodiment, the following effects can be obtained.

As depicted in FIG. 18A, the first annular electrode 282a is arranged near the central line C of the first high-frequency electrode ring 254 (see FIG. 16C) of the main body side holding section 222. Further, the contact area of the first annular electrode 282a with respect to the living tissue is set smaller than that of the second annular electrode 282b or the third annular electrode 282c. That is, an amount of energy supplied to the living tissue from the first annular electrode 282a is set smaller than an amount of energy supplied to the living tissue from the second annular electrode 282b or the third annular electrode 282c.

Then, the temperature distribution T_{R1} in the R₁ axis direction given to the living tissue from the main body side holding section 222 depicted in FIG. 18A can be uniformed from a position corresponding to a central part (near the central line C) and a position corresponding to an edge portion of the first high-frequency electrode ring 254 of the main body side holding section 222 as compared with the temperature distribution T_{R1} according to the prior art shown in FIG. 18B. That is, temperature gradient of the temperature distribution T_{R1} in the R₁ axis direction given to the living tissue from the main body side holding section 222 depicted in FIG. 18A can be further flattened with respect to temperature gradient of the temperature distribution T_{R1} of the prior art shown in FIG. 18B. Then, an arrangement of the electrodes 282a, 282b, and 282c in the R₁ axis direction of the main body side holding section 222 enables performing a further uniform treatment, e.g., weld or cautery with respect to the living tissue.

It is to be noted that each of the first and second high-frequency electrode rings 254 and 274 has the annular shape in this embodiment, but various kinds of shapes, e.g., an elliptic shape can be allowed.

### Ninth Embodiment

A ninth embodiment will now be explained with reference to FIG. 19. This embodiment is a modification of the eighth embodiment, and like reference numerals denote members equal to those explained in the eighth embodiment, thereby omitting a detailed explanation.

As shown in FIG. 19, a first high-frequency electrode ring 254 (see FIG. 16C) includes a first annular electrode 302a and a second annular electrode 302b. Of these electrodes, the first annular electrode (an inner electrode) 302a is arranged on an inner side of a central line C, and the second annular electrode (an outer electrode) 302b is arranged on an outer side of the first annular electrode 302a. Further, an annular insulating member 304 is arranged between the first annular electrode 302a and the second annular electrode 302b. It is to be noted that the central line C of the first high-frequency electrode ring 254 is present on the insulating member 304.

At this time, as shown in FIG. 19, a width in an R₁ axis direction of the first annular electrode 302a is substantially equal to a width in the R₁ axis direction of the second annular electrode 302b. Therefore, energy in the R₁ axis direction that is supplied to a living tissue from the first annular electrode 302a is substantially equal to energy in the R₁ axis direction that is given to the living tissue from the second annular electrode 302b.

On the other hand, the living tissue that is in contact with the first annular electrode 302a or the second annular electrode 302b is away from the central line C and close to the outside of a holding section 206. Hence, the living tissue is affected by the outside of the holding section 206 having temperature far lower than that of the living tissue provided between the main body side holding section 222 and the detachable side holding portion 224. Therefore, energy supplied to the living tissue from the first annular electrode 302a or the second annular electrode 302b is reduced due to an influence of the outside of the holding section 206.

Therefore, when the energy supplied to the living tissue held by the holding section 206 from the first annular electrode 302a or the second annular electrode 302b is adjusted, temperature distribution T_{R1} on a surface of the holding section 206 in the R₁ axis direction is further flattened.

### Tenth Embodiment

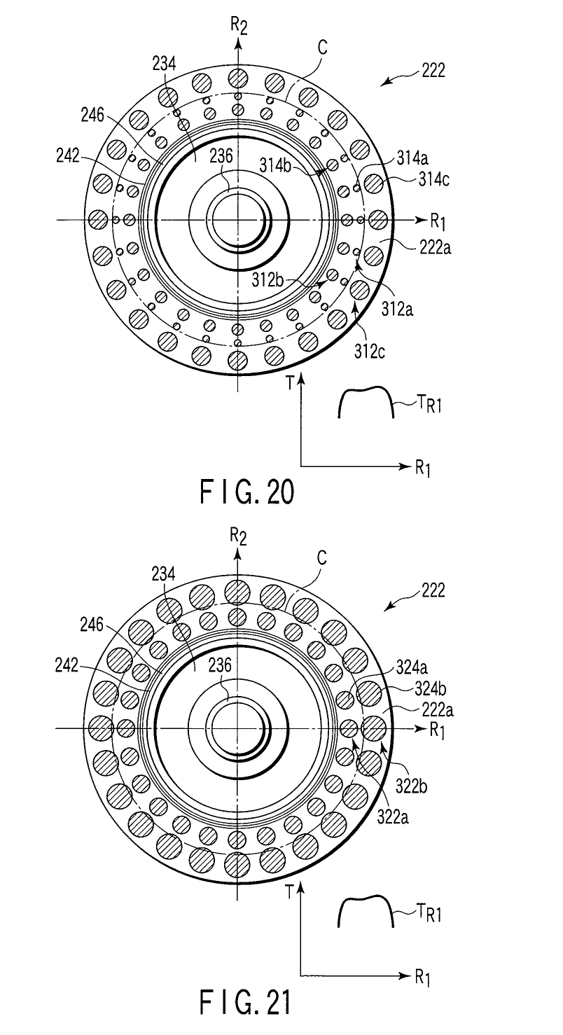

A tenth embodiment will now be explained with reference to FIG. 20. This embodiment is a modification of the eighth embodiment, and like reference numerals denote members equal to those explained in the eighth embodiment, thereby omitting a detailed explanation.

As shown in FIG. 20, a first high-frequency electrode ring 254 (see FIG. 16C) concentrically includes a first annular electrode group 312a, a second annular electrode group 312b, and a third annular electrode group 312c. Of these electrode groups, the first annular electrode group (a region near a central axis as a first region) 312a is arranged slightly inwards from a position near a central line C between an inner circumference and an outer circumference of the first high-frequency electrode ring 254. The second annular electrode group (a region away from the central axis as a second region) 312b is arranged on an inner side of the first annular electrode group 312a. The third annular electrode group (a region away from the central region as the second and/or third region) 312c is arranged on an outer side of the first annular electrode group 312a.

The first annular electrode group 312a includes a plurality of circular electrodes 314a on the same circumference. The second annular electrode group 312b includes a plurality of circular electrodes 314b on the same circumference. The third annular electrode group 312c includes a plurality of circular electrodes 314c on the same circumference. The electrodes 314a, the electrodes 314b, and the electrodes 314c are aligned in a radial direction, e.g., an R₁ axis direction and an R₂ axis direction. That is, each of the first to third annular electrode groups 312a, 312b, and 312c includes the same number of the electrodes 314a, 314b, or 314c having the same central angle. Therefore, a length of an arc between centers of the respective electrodes 314a in the first annular electrode group 312a (an intercentral distance) is longer than a length of an arc between centers of the respect electrodes 314b in the second annular electrode group 312b. Further, the length of the arc between the centers of the respective electrodes 314a in the first annular electrode group 312a is shorter than a length of an arc between centers of the respective electrodes 314c in the third annular electrode group 312c.

Here, comparing an area or a width in the R₁ axis direction (a diameter) of the electrode 314a in the first annular electrode group 312a with that of the electrode 314b in the second annular electrode group 312, the area or the diameter of the electrode 314b in the second annular electrode group 312b is larger. Comparing the area or the diameter of the electrode 314b in the second annular electrode group 312b with that of the electrode 314c in the third annular electrode group 312c, the area or the diameter of the electrode 314c in the third annular electrode group 312c is larger.

Therefore, density of the second annular electrode group 312b is higher than density of the third annular electrode group 312c, but the area or the diameter of each electrode 314b in the second annular electrode group 312b is smaller than that of each electrode 314c in the third annular electrode group 312c. Therefore, amounts of energies supplied to the living tissue from the second annular electrode group 312b and the third annular electrode group 312c are balanced. Then, even though a heating value near the central line C given from the first annular electrode group 312a is small, it is further flattened.

Therefore, the living tissue is uniformly treated in the R₁ axis direction of the holding section 206. Accordingly, when, e.g., welding the living tissue, the living tissue is uniformly cauterized, thereby obtaining uniform conjugation strength.

It is to be noted that the example where the first annular electrode group 312a includes the plurality of electrodes 314a has been explained in this embodiment, but a structure where the first annular electrode group 312a is formed into a continuous annular shape like the first annular electrode 282a (see FIG. 17) explained in the eighth embodiment is also preferable.

### Eleventh Embodiment

An eleventh embodiment will now be explained with reference to FIG. 21. This embodiment is a modification of the eighth to tenth embodiments, and like reference numerals denote members equal to those explained in the eighth to tenth embodiment, thereby omitting a detailed explanation.

As shown in FIG. 21, a first high-frequency electrode ring 254 (see FIG. 16C) includes a first annular electrode group 322a and a second annular electrode group 322b. Of these electrode groups, the first annular electrode group (an inner electrode group) 322a is arranged on an inner side, and the second electrode group (an outer electrode group) 322b is arranged on an outer side of the first annular electrode group 322a. The first annular electrode group 322a includes a plurality of circular electrodes 324a on a circumference. The second annular electrode group 322b includes a plurality of circular electrodes 324b on a circumference.

These first and second annular electrode groups 322a and 322b are arranged on two circumferences along a radial direction, e.g., an R₁ axis direction and an R₂ axis direction. That is, the first and second annular electrode groups 322a and 322b respectively include the same number of electrodes 324a and 324b. Therefore, a length of an arc between centers of the respective electrodes 324a (an intercentral distance) in the first annular electrode group 322a is shorter than a length of an arc between centers of the respective electrodes 324b in the second annular electrode group 322b.

Here, comparing an area or a width in the R₁ axis direction (a diameter) of each electrode 324a in the first annular electrode group 322a with that of each electrode 324b in the second annular electrode group 322b, the area or the diameter of each electrode 324b in the second annular electrode group 322b is larger.

Therefore, energy in the R₁ axis direction supplied to a living tissue from the first annular electrode group 322a is substantially equal to that from the second annular electrode group 322b.

On the other hand, the living tissue that is in contact with the inner side of the first annular electrode group 322a or the outer side of the second annular electrode group 322b is away from a central line C and close to the outside of a holding section 206. Hence, the living tissue is affected by the outside of the holding section 206 having temperature far lower than that of the living tissue present between a holding section 222 on a main body side and a detachable side holding portion 224. Therefore, energy supplied to the living tissue by the first annular electrode group 322a or the second annular electrode group 322b is reduced due to an influence of the outside of the holding section 206.

Accordingly, temperature distribution T_{R1} on a surface of the living tissue held by the holding section in the R₁ axis direction of the holding section 206 is approximated to a further flat state by adjusting the energy that is supplied to the living tissue from the first annular electrode group 322a or the second annular electrode group 322b while considering an influence of the outside of the holding section 206 having low temperature.

### Twelfth Embodiment

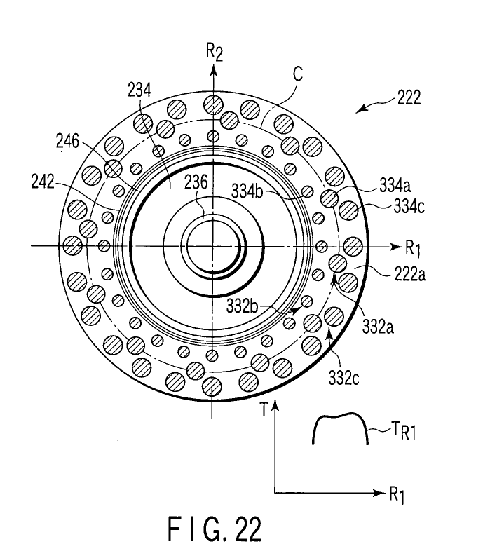

A twelfth embodiment will now be explained with reference to FIG. 22. This embodiment is a modification of the tenth embodiment, and like reference numerals denote members equal to those explained in conjunction with the tenth embodiment, thereby omitting a detailed explanation.

Respective a second annular electrode group and a third annular electrode group shown in FIG. 22 likewise have a second annular electrode group 312b and a third annular electrode group 312c shown in FIG. 20. Here, for accommodation, a reference numeral 332b denotes as the second annular electrode group and a reference numeral 332c denotes as the third annular electrode group. Further, a reference numeral 332a denotes as a first annular electrode group.

Furthermore, respective electrodes of the second annular electrode group 332b and electrodes of the third annular electrode group 332c shown in FIG. 22 likewise have electrodes 314b of the second annular electrode group 312b and electrodes 314c of the third annular electrode group 312c shown in FIG. 20. Here, for accommodation, a reference numeral 334b denotes as the electrodes of the second annular electrode group 332b and a reference numeral 334c denotes as the electrodes of the third annular electrode group 332c. Further, a reference numeral 332a denotes as electrodes of the first annular electrode group 332a.

The first annular electrode group 332a is arranged near the central line C of the first high-frequency electrode ring 254 (see FIG. 16C) between an inner side and an outer side.

The first annular electrode group 332a includes a plurality of circular electrodes 334a on a circumference. The number of the electrodes 334a in this embodiment is reduced to ½ of that of the electrodes 334a depicted in FIG. 20. However, a diameter of each electrode 334a is formed to be larger than that of each electrode 334a depicted in FIG. 20 to cancel out this reduction.

Therefore, a length of an arc between centers of the respective electrodes 334a (an intercentral distance) in the first annular electrode group 332a is longer than a length of an arc between the respective electrodes 334b in the second annular electrode group 332b. Further, the length of the arc between the centers of the respective electrodes 334a in the first annular electrode group 332a is longer than a length of an arc between centers of the respective electrodes 334c in the third annular electrode group 332c.

Here, comparing an area or a width in an R₁ axis direction (a diameter) of each electrode 334a in the first annular electrode group 332a with that of each electrode 334b in the second annular electrode group 332b, the area or the diameter of each electrode 334a in the first annular electrode group 332a is larger than the area or the diameter of each electrode 334b in the second annular electrode group 332b. Comparing the area or the diameter of each electrode 334a in the first annular electrode group 332a with that of each electrode 334c in the third annular electrode group 332c, the area or the diameter of each electrode 334c in the third electrode group 332c is equal to or larger than the area or the diameter of each electrode 334a in the first annular electrode group 332a.

Therefore, density of the second annular electrode group 332b is higher than that of the third annular electrode group 332c, the area or the diameter of each electrode 334b in the second annular electrode group 332b is smaller than that of each electrode 334c in the third annular electrode group 332c. Therefore, amounts of energies supplied to a living tissue from the second annular electrode group 332b and the third annular electrode group 332c are balanced. Then, even though a heating value near the central line C that is given from the first annular electrode group 312a is small, it is further flattened.

Accordingly, the living tissue is uniformly treated in the R₁ axis direction of a holding section 206. Therefore, when, e.g., welding the living tissue, the living tissue is uniformly cauterized, thereby obtaining uniform conjugation strength.

It is to be noted that the example where each of the electrodes 314a, 314b, 314c, 334a, 334b and 334c has the circular shape has been explained in the tenth to twelfth embodiments, but various kinds of shapes, e.g., an elliptic shape or a rhombic shape can be allowed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A treatment system that applies energy to a living tissue, comprising:
first and second holding members each having a holding surface to hold the living tissue;
an operating section that operates a relative movement of at least one of the first and second holding members with respect to the other;
an energy source that supplies energy to at least one of the first and second holding members; and
a plurality of energy applying portions that apply energy supplied from the energy source, the plurality of energy applying portions being provided on the holding surface of at least one of the first and second holding members and uniformly controlling density of energy applied to the living tissue held by the first and second holding members.

2. The treatment system according to claim 1,
wherein each of the first and second holding members includes a proximal end, a distal end, and a central axis in a longitudinal direction,
the plurality of energy applying portions include a first region arranged near the central axis of the holding surface of at least one of the first and second holding members, and a second region arranged at a position away from the central axis, and
energy density of the energy applying portions in the first region is smaller than energy density of the energy applying portions in the second region.

3. The treatment system according to claim 2,
wherein the plurality of energy applying portions are arranged like spots in each of the first and second regions, and
the number of the energy applying portions arranged in the first region is fewer than the number of the energy applying portions arranged in the second region.

4. The treatment system according to claim 2,
wherein the plurality of energy applying portions are arranged like spots in each of the first and second regions, and
a gap between the energy applying portions arranged in the first region is larger than a gap between the energy applying portions arranged in the second region.

5. The treatment system according to claim 2,
wherein the plurality of energy applying portions are arranged like spots in each of the first and second regions, and
an area of each energy applying portion arranged in the first region is smaller than an area of each energy applying portion arranged in the second region.

6. The treatment system according to claim 1,
wherein each of the first and the second holding members includes a proximal end, a distal end, and a central axis in a direction perpendicular to the longitudinal direction,
the plurality of energy applying portions include a region near the central axis that is arranged near the central axis of the holding surface of at least one of the first and second holding members, and a region away from the central axis that is arranged at a position away from the central axis, and
energy density of the energy applying portions in the region near the central axis is smaller than energy density of the energy applying portions in the region away from the central axis.

7. The treatment system according to claim 6,
wherein the plurality of energy applying portions are arranged like spots in each of the region near the central axis and the region away from the central axis, and
the number of the energy applying portions arranged in the region near the central axis is fewer than the number of the energy applying portions arranged in the region away from the central axis.

8. The treatment system according to claim 6,
wherein the plurality of energy applying portions are arranged like spots in each of the region near the central axis and the region away from the central axis, and
a gap between the energy applying portions arranged in the region near the central axis is larger than a gap between the energy applying portions arranged in the region away from the central axis.

9. The treatment system according to claim 6,
wherein the plurality of energy applying portions are arranged like spots in each of the region near the central axis and the region away from the central axis, and
an area of each energy applying portion arranged in the region near the central axis is smaller than an area of each energy applying portion arranged in the region away from the central axis.

10. The treatment system according to claim 1,
wherein each of the first and the second holding members has an annular shape having an inner circumference, an outer circumference, and a central line between the inner circumference and the outer circumference,
the plurality of energy applying portions include a first region arranged near the central line, a second region separated inwards from the central line, and a third region separated outwards from the central line, and
energy density of the energy applying portions in the first region is smaller than energy density of the energy applying portions in each of the second and third regions.

11. The treatment system according to claim 10,
wherein a width in a radial direction of each energy applying portion in the first region is smaller than a width in the radial direction of each energy applying portion in each of the second and third regions.

12. The treatment system according to claim 10,
wherein the plurality of energy applying portions arranged in the first to third regions are respectively concentrically arranged, and
an area of each energy applying portion arranged in the first region is smaller than an area of each energy applying portion arranged in each of the second and third regions.

13. The treatment system according to claim 10,
wherein the plurality of energy applying portions arranged in the first to third regions are respectively concentrically arranged,
a gap between the plurality of energy applying portions adjacent to each other on the same circumference in the first region is larger than a gap between the plurality of energy applying portions adjacent to each other on the same circumference in the second region,
the gap between the plurality of energy applying portions adjacent to each other on the same circumference in the first region is smaller than a gap between the plurality of energy applying portions adjacent to each other on the same circumference in the third region, and
an area of each energy applying portion arranged in the first region is smaller than an area of each energy applying portion arranged in the second and third regions.

14. The treatment system according to claim 10,
wherein the plurality of energy applying portions are arranged like spots in each of the first to third regions, and
the number of the energy applying portions arranged in the first region is fewer than the number of the energy applying portions arranged in each of the second and third regions.

15. The treatment system according to claim 10,
wherein the plurality of energy applying portions are arranged like spots in each of the first to third regions, and
a gap between the energy applying portions arranged in the first region is larger than a gap between the energy applying portions arranged in each of the second and third regions.

16. The treatment system according to claim 10,
wherein the plurality of energy applying portions are arranged like spots in each of the first to third regions, and
an area of each energy applying portion arranged in the first region is smaller than an area of each energy applying portion arranged in each of the second and third regions.

17. A treatment device that allows energy to function with respect to a living tissue, comprising:
a holding section that holds the living tissue, the holding section including:
first and second holding members that are relatively movable with respect to each other; and
a plurality of energy applying portions that are provided on at least one of the first and second holding members and connected with an energy source, the energy applying portions being provided on at least one of the first and second holding members and uniforming density of energy applied to the living tissue when applying the energy to the living tissue held by the first and second holding members.

18. The treatment device according to claim 17,
wherein each of the first and the second holding members includes a proximal end, a distal end, a central axis in a longitudinal direction, and a holding surface arranged at a position close to the other holding member,
the plurality of energy applying portions include a first region arranged near the central axis on the holding surface of at least one of the first and second holding members, and second and third regions arranged at positions away from the central axis, and
energy density of the energy applying portions in the first region is smaller than energy densities of the energy applying portions in each of the second and third regions.

19. The treatment device according to claim 18,
wherein the plurality of energy applying portions are arranged like spots in each of the first to third regions, and
the number of the energy applying portions arranged in the first region is fewer than the number of the energy applying portions arranged in each of the second and third regions.

20. The treatment device according to claim 18,
wherein the plurality of energy applying portions are arranged like spots in each of the first to third regions, and
a gap between the energy applying portions arranged in the first region is larger than a gap between the energy applying portions arranged in each of the second and third regions.

21. The treatment device according to claim 18,
wherein the plurality of energy applying portions are arranged like spots in each of the first to third regions, and
an area of each energy applying portion arranged in the first region is smaller than an area of each energy applying portion arranged in each of the second and third regions.

22. The treatment device according to claim 17,
wherein each of the first and the second holding members includes a proximal end, a distal end, and a central axis in a direction perpendicular to a longitudinal direction,
the plurality of energy applying portions include a region near the central axis that is arranged near the central axis of the holding surface of at least one of the first and second holding members, and a region away from the central axis that is arranged at a position away from the central axis, and
energy density of the energy applying portions in the region near the central axis is smaller than energy density of the energy emitting portions in the region away from the central axis.

23. The treatment device according to claim 22,
wherein the plurality of energy applying portions are arranged like spots in each of the region near the central axis and the region away from the central axis, and
the number of the energy applying portions arranged in the region near the central axis is fewer than the number of the energy applying portions arranged in the region away from the central axis.

24. The treatment device according to claim 22,
wherein the plurality of energy applying portions are arranged like spots in each of the region near the central axis and the region away from the central axis, and
a gap between the energy applying portions arranged in the region near the central axis is larger than a gap between the energy applying portions arranged in the region away from the central axis.

25. The treatment device according to claim 22,
wherein the plurality of energy applying portions are arranged like spots in each of the region near the central axis and the region away from the central axis, and
an area of each energy applying portion arranged in the region near the central axis is smaller than an area of each energy applying portion arranged in the region away from the central axis.

26. The treatment device according to claim 17,
wherein each of the first and the second holding members has an annular shape having an inner circumference, an outer circumference, and a central line between the inner circumference and the outer circumference,
the energy applying portions include a first region arranged near the central line, a second region separated inwards from the central line, and a third region separated outwards from the central line, and
energy density of the energy applying portions in the first region is smaller than energy density of the energy applying portions in each of the second and third regions.

27. The treatment device according to claim 26,
wherein a width in a radial direction of each energy applying portion in the first region is smaller than a width in the radial direction of each energy applying portion in each of the second and third regions.

28. The treatment device according to claim 26,
wherein the plurality of energy applying portions arranged in the first to third regions are respectively concentrically arranged, and
an area of each energy applying portion arranged in the first region is smaller than an area of each energy applying portion arranged in each of the second and third regions.

29. The treatment device according to claim 26,
wherein the plurality of energy applying portions arranged in the first to third regions are respectively concentrically arranged,
a gap between the plurality of energy applying portions adjacent to each other on the same circumference in the first region is larger than a gap between the plurality of energy applying portions adjacent to each other on the same circumference in the second region,
the gap between the plurality of energy applying portions adjacent to each other on the same circumference in the first region is smaller than a gap between the plurality of energy applying portions adjacent to each other on the same circumference in the third region, and
an area of each energy applying portion arranged in the first region is smaller than an area of each energy applying portion arranged in the second and third regions.

30. The treatment device according to claim 26,
wherein the plurality of energy applying portions are arranged like spots in each of the first to third regions, and
the number of the energy applying portions arranged in the first region is fewer than the number of the energy applying portions arranged in each of the second and third regions.

31. The treatment device according to claim 26,
wherein the plurality of energy applying portions are arranged like spots in each of the first to third regions, and
a gap between the energy applying portions arranged in the first region is larger than a gap between the energy applying portions arranged in each of the second and third regions.

32. The treatment device according to claim 26,
wherein the plurality of energy applying portions are arranged like spots in each of the first to third regions, and
an area of each energy applying portion arranged in the first region is smaller than an area of each energy applying portion arranged in each of the second and third regions.

33. A treatment method for a living tissue using energy, comprising:
holding the living tissue;
applying energy to the living tissue and denaturing the living tissue; and
uniforming energy density at a desired position where the held living tissues denatures by the energy applied to the living tissue.
